(19) United States
(12) Patent Application Publication
(10) Pub. No.: US 2025/0338814 A1
(43) Pub. Date: Nov. 6, 2025

(54) ACEROLA CHERRY VARIETY DESIGNATED NUTRILITE ACEROLA SUPER C

(71) Applicant: ACCESS BUSINESS GROUP INTERNATIONAL LLC, Ada, MI (US)

(72) Inventors: Ana Talita ADEODATO CARVALHO PONTES, Ubajara (BR); Joaquim Alex RODRIGUES DURAN, Tiangua (BR); Jomar Patricio MONTEIRO, Sobral (BR); Magaly Sales MONTEIRO, Sobral (BR)

(73) Assignee: ACCESS BUSINESS GROUP INTERNATIONAL LLC, Ada, MI (US)

(21) Appl. No.: 18/975,934

(22) Filed: Dec. 10, 2024

Related U.S. Application Data
(63) Continuation of application No. 18/431,749, filed on Feb. 2, 2024, now abandoned.
Application No. 18/431,749 is a continuation of application No. 16/890,561, filed on Jun. 2, 2020, now abandoned.
(60) Provisional application No. 62/857,888, filed on Jun. 6, 2019.
(60) Provisional application No. 62/986,197, filed on Mar. 6, 2020.

(51) Int. Cl. A01H 6/74; A01H 5/08

(52) U.S. Cl. CPC A01H 6/7445; A01H 5/08

(57) ABSTRACT

Described herein is an acerola cherry (Malpighia emarginata DC.) variety designated Nutrilite Acerola Super C, plants thereof, seed thereof, hybrids thereof, products thereof, and cultivars derived therefrom. The Nutrilite Acerola Super C variety has greater fruit yield, greater fruit mass, greater productivity, greater amounts of vitamin C, greater amounts of polyphenols, greater amounts of antioxidants, improved fruit firmness, and greater juice yields, among other desirable phenotypic or genotypic characteristics, as compared to other varieties of acerola plants or wild-type acerola.

### RELATED APPLICATIONS

The present patent document is a continuation application of U.S. patent application Ser. No. 18/431,749, filed Feb. 2, 2024, which is a continuation application of U.S. patent application Ser. No. 16/890,561, filed Jun. 2, 2020, which claims the benefit of the filing date under 35 U.S.C. § 119 (e) of Provisional U.S. Patent Application Ser. No. 62/857,888, filed Jun. 6, 2019, and Provisional U.S. Patent Application Ser. No. 62/986,197, filed Mar. 6, 2020, which are hereby incorporated by reference.

### REFERENCE TO APPENDIX [CD ROM/SEQUENCE LISTING]

The instant application contains a Sequence Listing XML which has been submitted electronically in XML format and is hereby incorporated by reference in its entirety. Said XML copy, created on Feb. 2, 2024, is named “503086_5002852_SEQ_LISTING.xml” and is 9,785 bytes in size.

### TECHNICAL FIELD

Described herein is an acerola cherry (Malpighia emarginata DC.) variety designated Nutrilite Acerola Super C, plants thereof, seed thereof, hybrids thereof, products thereof, and cultivars derived therefrom. The Nutrilite Acerola Super C variety has greater fruit yield, greater fruit mass, greater productivity, greater amounts of vitamin C, greater amounts of polyphenols, greater amounts of antioxidants, improved fruit firmness, and greater juice yields, among other desirable phenotypic or genotypic characteristics, as compared to other varieties of acerola plants or wild-type acerola.

### BACKGROUND

Acerola cherries (Malpighia emarginata DC.), also known as Barbados or West Indian cherries, are excellent sources of vitamin C and antioxidants (Franca, 2016). Acerola is believed to have originated in the Yucatan peninsula and is distributed from South Texas through Mexico and Central America to northern South America and throughout the Caribbean (Johnson et al., 2003). Acerola has been successfully grown in sub-tropical areas throughout the world, and some of the largest plantings are in Brazil. Acerola has great potential for expansion, since its potential utilization includes several markets, such as nutraceuticals, vitamins, juices, or pharmaceuticals (Almeida et al., 2014). In Brazil, commercial orchards are located in several states, but the northeast region is the largest producer due to its soil and climate conditions; the northeast produces 70% of the national production whereas the Southeast produces approximately 15% of the national production (Furlaneto and Nasser, 2015).

Acerola is a diploid and primarily allogamous species. There is high genetic variability in the morphological and quantitative characteristics (Mondin et al., 2010). The plant is a bushy shrub or small tree of about 6 m in height with a short trunk and spreading and drooping branches. The evergreen leaves are elliptic, oblong, or obovate. Immature plant leaves have white, silky, irritating hairs; the leaves become green and glossy when mature. The flowers have five pink or lavender spoon-shaped petals. The fruits are three-lobed 1.25-2.5 cm wide oblate drupes with glossy bright-red skin and juicy orange-colored acidic pulp. Each fruit contains three imbricate flattened seeds that each have two large fluted wings and one small wing that form triangular inedible “stones” (Morton, 1987). Acerola's main pollinators are small insects and Centris bees.

Genetic variability is significant for the improvement of many crop species including acerola. Acerola has great genetic variability due to the heterogeneity of the orchards in special commercial plots originated from seed propagation (Moraes Filho et al., 2013). Orchards having high genetic diversity typically have lower commercial yields. However, the occurrence of genetic variability promoted by seed propagation has permitted the identification and selection of genotypes with interesting commercial traits (Salla et al., 2002).

In breeding programs, the study of phenotypic traits in perennial plants, such as acerola, is essential for the selection process (Fachi et al., 2016). However, biotechnology tools can also be used to accelerate the acerola breeding programs, such as the use of markers to estimate genetic variability among clones and to indicate the most divergent clones for crosses (Lima et al., 2015).

There are only a few varieties of acerola available to Brazilian producers and this limits the crop's potential. According to the Brazilian Ministry of Agriculture, Livestock, and Food Supply, only 14 acerola varieties are registered (Brazilian Ministry of Agriculture, Livestock, and Food Supply, 2018). This demonstrates that despite the high genetic variability found in Brazilian orchards, there are only a small number of varieties. In addition, few studies have evaluated, identified, and selected varieties with agriculturally advantageous traits to improve acerola crop production.

Previous efforts have primarily focused on developing domesticated acerola varieties with high yields, high vitamin C, disease resistance, or drought tolerance. Few efforts have been made to generate acerola varieties that have high vitamin C and enhanced yields among other agriculturally advantageous traits. Therefore, there is a need to develop new acerola (Malpighia emarginata DC.) varieties that have greater fruit yield, greater fruit mass, greater productivity, greater amounts of vitamin C, greater amounts of polyphenols, greater amounts of antioxidants, improved fruit firmness, greater juice yields, among other desirable phenotypic or genotypic characteristics, as compared to other varieties of acerola plants or wild-type acerola.

### SUMMARY

One embodiment described herein is an acerola cherry (Malpighia emarginata DC.) variety designated Nutrilite Acerola Super C, a representative sample of seed having been deposited under ATCC Patent Deposit Designation, Patent Deposit No. PTA-126522. In one aspect, the cherry comprises one or more of the characteristics: fruit firmness of about 8 to 12 N; juice yield of about 50-70%; vitamin C content of about 3000-3500 mg·100 g⁻¹; total soluble phenols of about 4000-4500 mg GAE·100 g⁻¹; or total antioxidant activity of about 300-350 μmol TEAC·g⁻¹ pulp. In another aspect, the cherry fruit has an average mass of about 4.4 g.

Another embodiment is a plant product produced from one or more of the acerola cherries of described herein comprising a cell or the genetic material of the cherry. In one aspect, the plant product comprises fruit, juice, pulp, extracts, vitamin C, polyphenols, antioxidants, derivatives thereof, or combinations thereof. Another aspect is acerola cherry juice or pulp produced from one or more of the acerola cherries of described herein comprising a cell or the genetic material of the cherry. Another aspect is a food product, health supplement, or nutraceutical produced from one or more of the acerola cherries described herein comprising a cell or the genetic material of the cherry. Another aspect is a food product comprising the acerola cherries described herein or a plant product thereof comprising a cell or the genetic material of the cherry.

Another embodiment described herein is a seed of the acerola cherries described herein comprising a cell or the genetic material of the cherry.

Another embodiment described herein is an acerola plant, or part thereof, produced by growing the cherry described herein comprising a cell or the genetic material of the cherry. In one aspect, the acerola plant is produced by natural plant breeding and vegetative propagation. In another aspect, the acerola plant yields about 25 to about 60 kg·tree⁻¹ or about 15 to about 36 metric tons of fruit per hectare. Another aspect is a homogenous population comprising a plurality of the acerola plant described herein. In another aspect, the acerola plant comprises or confers to its seed one or more traits comprising greater fruit yield, greater fruit mass, greater productivity, greater amounts of vitamin C, greater amounts of polyphenols, greater amounts of antioxidants, improved fruit firmness, greater juice yields, increased protein yield, increased fiber yield, increased fertilizer utilization, improved adaptation to winter planting, increased drought tolerance, increased cold tolerance, increased photoperiod, herbicide tolerance, increased insect resistance, increased disease resistance, or combinations thereof, as compared to other varieties of acerola plants or wild-type acerola.

Another embodiment described herein is a tissue culture of cells produced from the acerola plant described herein, wherein the cells are produced from a plant part comprising embryo, meristematic cell, leaf, cotyledon, hypocotyl, root, root tip, stem, pistil, anther, ovule, flower, pollen, or seed.

Another embodiment is an acerola cherry (Malpighia emarginata DC.) plant regenerated from a tissue culture, wherein the acerola plant comprises the morphological and physiological characteristics of variety Nutrilite Acerola Super C, a representative sample of seed having been deposited under ATCC Patent Deposit Designation, Patent Deposit No. PTA-126522.

Another embodiment described herein is an acerola cherry (Malpighia emarginata DC.) plant or plant part of the variety designated Nutrilite Acerola Super C, a representative sample of seed having been deposited under ATCC Patent Deposit Designation, Patent Deposit No. PTA-126522. Another aspect is a homogenous population comprising a plurality of the acerola plants described herein, wherein the population yields about 25 to about 60 kg·tree⁻¹ or about 15 to about 36 metric tons of fruit per hectare. Another aspect is fruit produced from the acerola plant described herein comprising a cell or the genetic material of the cherry plant. In one aspect, the fruit comprises one or more of the characteristics: fruit firmness of about 8 to 12 N; juice yield of about 50-70%; vitamin C content of about 3000-3500 mg·100 g⁻¹; total soluble phenols of about 4000-4500 mg GAE·100 g⁻¹; or total antioxidant activity of about 300-350 μmol TEAC·g⁻¹ pulp. In another aspect, the fruit has an average mass of at least about 4.4 g. In another aspect, the acerola plant or plant part comprises one or more traits comprising greater fruit yield, greater fruit mass, greater productivity, greater amounts of vitamin C, greater amounts of polyphenols, greater amounts of antioxidants, improved fruit firmness, greater juice yields, increased protein yield, increased fiber yield, increased fertilizer utilization, improved adaptation to winter planting, increased drought tolerance, increased cold tolerance, increased photoperiod, herbicide tolerance, increased insect resistance, increased disease resistance, or combinations thereof, as compared to other varieties of acerola plants or wild-type acerola. Another aspect is a descendant of the acerola plant described herein, wherein the descendant comprises one or more traits comprising greater fruit yield, greater fruit mass, greater productivity, greater amounts of vitamin C, greater amounts of polyphenols, greater amounts of antioxidants, improved fruit firmness, greater juice yields, increased protein yield, increased fiber yield, increased fertilizer utilization, improved adaptation to winter planting, increased drought tolerance, increased cold tolerance, increased photoperiod, herbicide tolerance, increased insect resistance, increased disease resistance, or combinations thereof, as compared to other varieties of acerola plants or wild-type acerola.

Another embodiment described herein is a germplasm of acerola cherry (Malpighia emarginata DC.) variety designated Nutrilite Acerola Super C, a representative sample of seed having been deposited under ATCC Patent Deposit Designation, Patent Deposit No. PTA-126522. One aspect is an acerola plant comprising the acerola germplasm of claim 24. Another aspect is fruit produced by the acerola plant described, comprising the acerola germplasm. In another aspect, the fruit comprises one or more of the characteristics: fruit firmness of about 8 to 12 N; juice yield of about 50-70%; vitamin C content of about 3000-3500 mg·100 g⁻¹; total soluble phenols of about 4000-4500 mg GAE·100 g⁻¹; or total antioxidant activity of about 300-350 μmol TEAC·g⁻¹ pulp. In another aspect, the fruit has an average mass of about 4.4 g. Another aspect is a homogenous population comprising a plurality of the acerola plants described herein, wherein the population yields about 25 to about 60 kg·tree⁻¹ or about 15 to about 36 metric tons of fruit per hectare.

Another embodiment described herein is a method for producing an acerola cherry, the method comprising: (a) planting a seed of acerola cherry (Malpighia emarginata DC.) variety designated Nutrilite Acerola Super C, a representative sample of seed having been deposited under ATCC Patent Deposit Designation, Patent Deposit No. PTA-126522, in pollinating proximity to itself or to a seed from a different acerola variety; (b) growing a plant from the seeds planted in pollinating proximity; and (c) harvesting one or more resultant acerola cherries. In one aspect, the acerola cherry comprises one or more of the characteristics: fruit firmness of about 8 to 12 N; juice yield of about 50-70%; vitamin C content of about 3000-3500 mg·100 g⁻¹; total soluble phenols of about 4000-4500 mg GAE·100 g⁻¹; or total antioxidant activity of about 300-350 μmol TEAC·g⁻¹ pulp.

Another embodiment described herein is a method for producing an acerola cherry, the method comprising: (a) grafting a scion of an acerola cherry plant (Malpighia emarginata DC.) variety designated Nutrilite Acerola Super C, a representative sample of seed having been deposited under ATCC Patent Deposit Designation, Patent Deposit No. PTA-126522, onto an acerola rootstock; (b) growing the grafted plant; and (c) harvesting one or more resultant acerola cherries. In one aspect, the acerola cherry comprises one or more of the characteristics: fruit firmness of about 8 to 12 N; juice yield of about 50-70%; vitamin C content of about 3000-3500 mg·100 g⁻¹; total soluble phenols of about 4000-4500 mg GAE·100 g⁻¹; or total antioxidant activity of about 300-350 μmol TEAC·g⁻¹ pulp. In another aspect, the rootstock comprises acerola variety BRS 366 “Jaburu” or BRS 235 “Apodi.”

Another embodiment described herein is a method for producing acerola cherries (Malpighia emarginata DC.) variety Nutrilite Acerola Super C, the method comprising: (a) crossing an acerola cherry (Malpighia emarginata DC.) variety designated Nutrilite Acerola Super C, a representative sample of seed having been deposited under ATCC Patent Deposit Designation, Patent Deposit No. PTA-126522, with a plant of a second, different acerola cherry (Malpighia emarginata DC.) variety to produce F₁ acerola cherries; and (b) harvesting the F₁ acerola cherries. One aspect is an F₁ acerola cherry (Malpighia emarginata DC.) produced by the method described herein.

Another embodiment described herein is a method of introducing a desired trait into acerola cherry (Malpighia emarginata DC.) variety Nutrilite Acerola Super C, the method comprising: (a) crossing acerola cherry (Malpighia emarginata DC.) variety designated Nutrilite Acerola Super C, a representative sample of seed having been deposited under ATCC Patent Deposit Designation, Patent Deposit No. PTA-126522, with a plant of another acerola cherry (Malpighia emarginata DC.) variety that comprises a desired trait to produce F₁ progeny plants; (b) selecting one or more progeny plants that have the desired trait to produce selected progeny plants; (c) crossing the selected progeny plants with the Nutrilite Acerola Super C plants to produce backcross progeny plants; (d) selecting for backcross progeny plants that have the desired trait; and (e) repeating steps (c) and (d) three or more times in succession to produce fourth or higher backcross progeny plants that comprise the desired trait. One aspect is an acerola cherry (Malpighia emarginata DC.) plant produced by the method of described herein, wherein the plant comprises traits and physiological and morphological characteristics of acerola cherry (Malpighia emarginata DC.) variety Nutrilite Acerola Super C, a representative sample of seed having been deposited under ATCC Patent Deposit Designation, Patent Deposit No. PTA-126522. In another aspect, the acerola cherry (Malpighia emarginata DC.) plant produced by the method, wherein the desired trait comprises one or more of: greater fruit yield, greater fruit mass, greater productivity, greater amounts of vitamin C, greater amounts of polyphenols, greater amounts of antioxidants, improved fruit firmness, greater juice yields, increased protein yield, increased fiber yield, increased fertilizer utilization, improved adaptation to winter planting, increased drought tolerance, increased cold tolerance, increased photoperiod, herbicide tolerance, increased insect resistance, increased disease resistance, or combinations thereof, as compared to other varieties of acerola plants or wild-type acerola.

Another embodiment described herein is a method for selecting an acerola cherry (Malpighia emarginata DC.) plant comprising the traits and the physiological and morphological characteristics of acerola cherry (Malpighia emarginata DC.) variety Nutrilite Acerola Super C, a representative sample of seed having been deposited under ATCC Patent Deposit Designation, Patent Deposit No. PTA-126522, the method comprising: performing one or more cross pollination selections; and performing one or more vegetative propagations and selections; wherein the selection selects for one or more of greater fruit yield, greater fruit mass, greater productivity, greater amounts of vitamin C, greater amounts of polyphenols, greater amounts of antioxidants, improved fruit firmness, greater juice yields, increased protein yield, increased fiber yield, increased fertilizer utilization, improved adaptation to winter planting, increased drought tolerance, increased cold tolerance, increased photoperiod, herbicide tolerance, increased insect resistance, increased disease resistance, or combinations thereof, as compared to other varieties of acerola plants or wild-type acerola. One aspect is an acerola cherry plant, fruit, or seed produced from the acerola plant described herein.

### DESCRIPTION OF THE DRAWINGS

The patent or application file contains at least one drawing executed in color. Copies of this patent or patent application publication with color drawings will be provided by the Office upon request and payment of the necessary fee.

FIG. 1A shows a schematic of the selection process. Initially 20 genotypes were grown from seeds for 3 years until maturity and the fruit yields (kg·tree⁻¹) and vitamin C content (mg vitamin C per 100 g of pulp) were evaluated. The ten most productive genotypes were propagated by seeds, grown for 2 years, and the productivity and vitamin C yield were again evaluated. The six most productive genotypes from the second selection were propagated by grafting scions on to rootstock. These plants were grown for 2 years and then evaluated for fruit and vitamin C yields. The most productive genotype was identified, e.g., Genotype A or Nutrilite Acerola Super C.

FIG. 1B shows a graph illustrating the vitamin C yield (%, mg vitamin C per 100 g of pulp) and fruit yield (kg·tree⁻¹) for six acerola genotypes (A-F) and four cultivars (BRS 235; BRS 366; Flor Branca; Monami). Data are shown in Table 5.

FIG. 2A shows a random amplification of polymorphic DNA (RAPD) profile of six acerola genotypes and four cultivars using the random primer OPAX 02 (left side) and OPAX 05 (right side). M: molecular marker (DNA ladder 1 kb Plus, Invitrogen); 1-3 Monami; 4 6 BRS 366; 7-9 BRS 235; 10-12 Flor Branca; 13-15 Nutrilite Acerola Super C; 16-18 Genotype B; 19-21 Genotype C; 22-24 Genotype D; 25-27 Genotype E; 28-30 Genotype F; C Control.

FIG. 2B shows a UPGMA dendrogram of ten acerola genotypes based on seven random RAPD primer based on Jaccard's similarity coefficient. Gen. A: Nutrilite Acerola Super C.

FIG. 3A shows a 1-year old Nutrilite Acerola Super C acerola cherry tree with an open growth habit.

FIG. 3B shows a 3-year old Nutrilite Acerola Super C acerola cherry tree with a high branch density.

FIG. 4A shows a 1-year old Nutrilite Acerola Super C acerola cherry tree branch with a medium (2) pubescence.

FIG. 4B shows a 1-year old Nutrilite Acerola Super C acerola cherry tree branch with a short (3) internode length and a medium diameter (5).

FIG. 5A shows a Nutrilite Acerola Super C acerola cherry leaf having a medium length (5) and medium width (5).

FIG. 5B shows a Nutrilite Acerola Super C acerola cherry leaf having the widest part in the middle (2), a medium margin (3), no variegation (1), and an intense dark green color (4).

FIG. 6A shows a Nutrilite Acerola Super C acerola cherry flower with the anthers (a) and stigma (b) indicated. The anther and stigma are on the same level (2). The flower is strongly curved.

FIG. 6B shows a Nutrilite Acerola Super C acerola cherry flower with a close-up of the anthers.

FIG. 7 shows a Nutrilite Acerola Super C acerola cherry flower highlighting the petals. The petals have a medium margin (3) and a dark pink color (3).

FIG. 8A shows a Nutrilite Acerola Super C acerola cherry with a long (6) length (a), a large (7) diameter (b), a ratio of length (a) to diameter (b) of medium (2), a high weight (7) and a flattened shape (3).

FIG. 8B shows a Nutrilite Acerola Super C acerola cherry having a deep (3) furrow depth.

FIG. 8C shows a Nutrilite Acerola Super C acerola cherry having a medium (2) distal cavity depth (c), and a narrow (1) distal cavity width (d) a medium (2) depth peduncular cavity (b) and a medium (5) stem length.

FIG. 9A shows a Nutrilite Acerola Super C acerola cherry having a deep (3) peduncular cavity, an orange (2) pulp color, high (4) acidity, and medium (5) succulence.

FIG. 9B shows a Nutrilite Acerola Super C acerola cherry seed having a medium (2) size and a medium (2) color intensity.

### DETAILED DESCRIPTION

Described herein is an acerola cherry (Malpighia emarginata DC.) variety designated Nutrilite Acerola Super C that has greater fruit yield, greater fruit mass, greater productivity, greater amounts of vitamin C, greater amounts of polyphenols, greater amounts of antioxidants, and greater juice yields, among other desirable phenotypic or genotypic characteristics, as compared to other varieties of acerola plants or wild-type acerola.

The phrase “agriculturally advantageous trait,” “agriculturally advantageous characteristic,” “desirable phenotypic characteristic,” or “desirable genotypic characteristics” as used herein refers to one or more traits that confer a growth advantage, production enhancement, or commercial benefit. Such traits or characteristics include, but are not limited to, increased metabolic efficiency; greater photosynthetic capacity; increased or more rapid growth rate; greater fruit yield; greater fruit weight; modified plant architecture; herbicide resistance; reduced or increased height; reduced or increased branching; reduced or increased number of leaves; increased or decreased number of flowers; increased or decreased flower size; total biomass; increased or decreased days to flowering; increased or decreased days to maturity; increased harvest index; enhanced cold or frost tolerance; improved vigor; enhanced color; increased color uniformity; greater product uniformity; enhanced resistance to insects, predators, or disease; improved storage characteristics; enhanced yield; greater water optimization; greater tolerance to dehydration, water deficit conditions, or drought; better recovery from dehydration, water deficit conditions, or drought; increased root growth; increased lateral root formation; increased root branching; increased surface area of roots; increased root mass; more root hairs; increased nutrient or fertilizer uptake; increased micronutrient uptake; enhanced salt tolerance; enhanced resistance of plant tissue to decay; enhanced heavy metal tolerance; enhanced sweetness; improved texture; decreased phosphate content; increased germination; increased oil content; increased protein content; increased carbohydrate content; increased fiber content; improved starch composition; improved flower longevity; enhanced health and nutritional characteristics; production of novel oils or resins; production of novel proteins or peptides; production of novel carbohydrates; enhanced agronomic traits; enhanced heritability of any of the foregoing traits, or any other agronomically desirable or commercially advantageous traits or characteristics.

The term “backcrossing” as used herein refers to a process where a progeny plant is crossed back to one of the parental genotypes one or more times. For example, crossing a first generation hybrid F₁ with one of the parental genotypes of the F₁ hybrid, and then crossing a second-generation hybrid F₂ with the same parental genotype, and so forth. Backcrossing is typically used to introduce one or more locus conversions from one genetic background into another.

The phrase “open pollination progenies” as used herein refers to plants that are pollinated through naturally occurring means. These plants bear seeds that produce plants that are identical to the parent plant. Open pollinated plants are also very genetically diverse and can be more adaptable to local growing conditions. Open pollination can be carried out by an external means such as birds, insects, water, or wind or by self-pollination, which occurs when the male and female parts are contained in the same plant.

The term “breeding” as used herein refers to the genetic manipulation of living organisms.

The term “cell” as used herein refers a plant cell, whether isolated, in tissue culture, or incorporated in a plant or plant part.

The term “cross pollination” refers to fertilization by the union of two gametes from different plants.

The term “descendant” as used herein refers to any generation plant.

The term “derived from” as used herein, unless otherwise specified, indicates that a particular thing (e.g., plant, seed, etc.) or group of things has originated from the source specified, but has not necessarily been obtained directly from the specified source.

The term “Fₙ” as used herein refers to the filial generation, where the subscript n refers to the generation number, such as F₁, F₂, F₃, etc.

The terms “improved,” “increased,” “enhanced,” or “greater” as used herein refer to the heightening or bettering of a particular characteristic or trait as compared to other similar organisms or a wild-type organism. Typically, this is an agriculturally advantageous trait. Increased or greater can refer to quantifiable characteristics such as mass or numbers or the bettering of an agriculturally advantageous characteristic or trait. An “improved” characteristic may refer to an increase or decrease of a characteristic or trait as appropriate under the circumstances.

The term “plant” as used herein refers to a plant at any developmental stage, as well as any part or parts of a plant that may be attached to or separated from an intact plant.

The term “plant part” as used herein comprises organs, tissues, and cells of a plant. Plant parts comprise leaves, stems, shoots, petioles, roots, root tips, root caps, root hair, leaf hair, seed hair, xylem, phloem, parenchyma, endosperm, flowers, inflorescences, florets, peduncles, filaments, pedicles, anthers, pistils, stamen, sepal, receptacles, stigma, style, ovaries, ovules, pollen, spores, microspores, gametophytes, sporophytes, embryos, fruit, pods, seeds, grain, cotyledons, hypocotyls, epicotyls, calli, meristematic cells, companion cells, guard cells, protoplasts, tissues, cells, or any other organs, tissues, cells, subcellular components of a plant, or combinations thereof.

The term “plant product” as used herein refers to an agricultural or commercial product created from a plant, plant part, fruit, or seed. Non-limiting examples of plant products comprise flowers, pollen, leaves, vines, stalks, fruits, berries, vegetables, cucurbits, roots, tubers, cones, pods, seeds, beans, grains, kernels, hulls, meals, grits, flours, sugars, starches, vitamins, antioxidants, polyphenols, protein concentrates, protein isolates, waxes, oils, extracts, juices, concentrates, liquids, or syrups.

The term “progeny” as used herein refers to a first generation (F₁) plant.

The term “fruit (e.g., berry or cherry) per kilogram” as used herein refers to the number of fruits required to comprise 1 kilogram mass.

The term “vitamin C content” as used herein refers to the mass of vitamin C (ascorbic acid) in mg per 100 grams of acerola pulp (mg·100 g pulp⁻¹).

The term “vitamin C yield” as used herein refers to the amount of vitamin C (ascorbic acid) in mg per 100 grams of acerola pulp (mg·100 g pulp⁻¹) converted to a percentage. For example, 1000 mg vitamin C per 100 g of pulp is 10% vitamin C.

The term “total soluble phenols” as used herein refers to the mass of gallic acid equivalents (GAE) in mg per 100 grams of acerola pulp (mg of GAE·100 g pulp⁻¹).

The term “total antioxidant activity” or “TAA” as used herein refers to the quantity of trolox-equivalent antioxidant capacity (TEAC) in micromoles per gram of acerola pulp (μmol TEAC·g pulp⁻¹).

The term “productivity” as used herein refers to mass of fruit from a single tree (kg·tree⁻¹).

The term “fruit firmness” as used herein refers to the measurement of a fruit's firmness using a 2.5 mm cylindrical probe with the results expressed as Newtons (N). One Newton is 1 kg·m·s⁻².

The term “juice yield” as used herein refers to the mass percentage of juice extracted to total fruit (%). The difference in mass is the amount of pulp.

The term “fruit yield” as used herein refers to the kilograms of fruit produced in a hectare plot and is reported as kilograms per hectare (kg·ha⁻¹). A hectare is 100 Ares and is equivalent to 10,000 m² or about 2.47 acres.

The term “sibbing,” “sibbed,” or “sib crossing” as used herein refers to the pollinating of an emasculated plant with pollen from a sister plant.

The term “Nutrilite Acerola Super C” as used herein refers to the acerola cherry (Malpighia emarginata DC.) variety Nutrilite Acerola Super C a representative sample of seed having been deposited under ATCC Patent Deposit Designation, Patent Deposit No. PTA-126522. In some tables, descriptions, and drawings herein, Nutrilite Acerola Super C is referred to as “Genotype A.”

The term “relative yield” as used herein refers to the percent increase in the mass of fruit per hectare as compared to other varieties.

The term “wild-type” as used herein refers to the typical form of an organism or its genetic material, as it normally occurs, as distinguished from a selected organism. In one aspect, the wild-type is an undomesticated acerola cherry (Malpighia emarginata) plant (or population thereof) or a domesticated acerola cherry plant (i.e., a variety or cultivar) (or population thereof) that has not undergone selection for agriculturally advantageous traits.

The term “about” as used herein refers to any value that is within a variation of up to ±10% of the value modified by the term “about.”

The article “a” or “an” as used herein means “one or more” unless otherwise specified.

The term “or” can be conjunctive or disjunctive.

Terms such as “include,” “including,” “contain,” “containing,” “have,” “having,” and the like mean “comprise” or “comprising.”

Acerola cherry (Malpighia emarginata DC.) is an important and valuable crop for both food and agricultural products. A goal of acerola breeding is to develop acerola varieties that are genetically stable, high yielding, and have other agriculturally advantageous characteristics. Acerola plants with such agriculturally advantageous traits can be selected, generated, crossed with other desirable varieties, and selected for enhanced agriculturally advantageous traits and hybrid vigor.

One embodiment described herein is an acerola cherry (Malpighia emarginata DC.) variety designated Nutrilite Acerola Super C that has higher fruit yield, greater amounts of vitamin C, polyphenols, and antioxidants, and higher juice yields, compared to other typical acerola varieties or wild-type acerola. In one aspect, the acerola cherry (Malpighia emarginata DC) variety designated Nutrilite Acerola Super C comprises a acerola cherry plant or a acerola cherry seed, a representative sample of seed having been deposited under ATCC Patent Deposit Designation, Patent Deposit No. PTA-126522.

Another embodiment described herein is a method for selecting an acerola (Malpighia emarginata DC.) plant comprising greater fruit yield, greater fruit mass, greater productivity, greater amounts of vitamin C, greater amounts of polyphenols, greater amounts of antioxidants, improved fruit firmness, and greater juice yields. The Nutrilite Acerola Super C variety was produced solely using pollination, selecting, grafting, and selfing methods and did not encompass mutagenesis, transformation, or other molecular genetic manipulations. The Nutrilite Acerola Super C variety of acerola is considered a non-genetically modified organism (non-GMO).

Another embodiment described herein is a seed of acerola (Malpighia emarginata DC.) variety designated Nutrilite Acerola Super C.

Another embodiment described herein is an acerola cherry (Malpighia emarginata DC.) plant of the Nutrilite Acerola Super C variety comprising greater fruit yield, greater fruit mass, greater productivity, greater amounts of vitamin C, greater amounts of polyphenols, greater amounts of antioxidants, improved fruit firmness, and greater juice yields. In another aspect, the Nutrilite Acerola Super C variety further comprises one or more of the following characteristics: greater fruit yield, greater fruit mass, greater productivity, greater amounts of vitamin C, greater amounts of polyphenols, greater amounts of antioxidants, greater juice yields, improved fruit firmness, increased amounts of sugars, increased fruit uniformity, increased fruit yield per hectare, increased protein yield, increased fiber yield, increased fertilizer utilization, rapid growth, improved adaptation to winter planting, increased drought tolerance, increased cold tolerance, increased photoperiod, herbicide tolerance, increased insect resistance, increased disease resistance, or combinations thereof, as compared to other varieties of acerola cherry plants or wild type acerola cherry.

Another embodiment described herein is an acerola cherry (Malpighia emarginata DC.) plant of the Nutrilite Acerola Super C variety comprising a productivity of about 25 to about 60 kg·tree⁻¹ or about 15 to about 36 metric tons of fruit per hectare (assuming 600 plants hectare⁻¹). In one aspect, Nutrilite Acerola Super C variety has a productivity of about 25 kg·tree⁻¹ during the first year of production or about 15 metric tons per hectare and about 50-60 kg·tree⁻¹ or about 30-36 metric tons per hectare during the second and subsequent years of production. In one aspect, the Nutrilite Acerola Super C variety produces about 28.9 kg in the first year or about 17.3 metric tons of fruit per hectare. In another aspect, the Nutrilite Acerola Super C variety produces about 17.3 metric tons of fruit per hectare (assuming 600 plants hectare⁻¹) during the first year of production. In one aspect, the Nutrilite Acerola Super C variety produces about 50.6 kg in the second or subsequent years or about 30.3 metric tons of fruit per hectare. In another aspect, the Nutrilite Acerola Super C variety produces about 30.3 metric tons of fruit per hectare (assuming 600 plants hectare⁻¹) during the second and subsequent years of production.

Another embodiment described herein is an acerola cherry (Malpighia emarginata DC.) comprising cherries with a fruit firmness of about 8 to 12 N. In one aspect, the immature fruit has a firmness of about 9 N. In another aspect, the ripe fruit has a firmness of about 3 N.

Another embodiment described herein is an acerola cherry (Malpighia emarginata DC.) comprising cherries with a juice yield of about 50-70%.

Another embodiment described herein is an acerola cherry (Malpighia emarginata DC.) comprising cherries with a vitamin C (ascorbic acid) content of about 3000-3800 mg·100 g⁻¹.

Another embodiment described herein is an acerola cherry (Malpighia emarginata DC.) comprising cherries with a vitamin C (ascorbic acid) content of about 32%.

Another embodiment described herein is an acerola cherry (Malpighia emarginata DC.) comprising cherries with total soluble phenols of about 4000-4500 mg GAE·100 g⁻¹.

Another embodiment described herein is an acerola cherry (Malpighia emarginata DC.) comprising cherries with total antioxidant activity of about 300-350 μmol TEAC·g⁻¹ pulp.

Another embodiment described herein is an acerola cherry plant (Malpighia emarginata DC.) isolated by screening for genotypes comprising desirable agronomic characteristics. After the first screening, potential genotypes were selected and seeds obtained and planted to produce F₂ progeny. The F₂ progenies were again selected for desirable agronomic characteristics. The most desirable F₂ progenies were propagated by grafting to produce identical clones. Nutrilite Acerola Super C was selected from the most productive (vitamin C and fruit yield) grafted F₂ clones.

In one embodiment described herein, an acerola cherry plant (Malpighia emarginata DC.) Nutrilite Acerola Super C is obtained using a grafting method that involves joining parts of two plants to function as a single plant. One of the plants provides the root system, i.e., the rootstock. The other plant provides the scion or upper portion (branches or stems) that has the desirable agricultural characteristics of Nutrilite Acerola Super C. Grafting maintains the genetic characteristics and agronomic qualities of the parent and permits exact genetic duplicates of individual plants. Acerola seedlings produced by seeds, although similar to the parent in the yield and fruit quality, may have slightly different genetic characteristics due to segregation and recombination.

Another embodiment described herein is an acerola cherry (Malpighia emarginata DC.) plant variety designated Nutrilite Acerola Super C. In one aspect, the plant can be obtained by planting a seed of acerola (Malpighia emarginata DC.) variety designated Nutrilite Acerola Super C in a growth medium and growing the plant. The medium can comprise soil, artificial soil, compost, culture media, hydroponic or hydroculture media, or any other suitable medium for growing plants.

Another embodiment described herein is an acerola cherry (Malpighia emarginata DC.) plant variety designated Nutrilite Acerola Super C. In one aspect, the plant can be obtained by grafting a scion of acerola (Malpighia emarginata DC.) variety designated Nutrilite Acerola Super C onto a rootstock and growing the plant in a growth medium. The medium can comprise soil, artificial soil, compost, culture media, hydroponic or hydroculture media, or any other suitable medium for growing plants. In one aspect, the rootstock is BRS 366 “Jaburu” or BRS 235 “Apodi.” Another aspect described herein is an acerola cherry plant produced by the foregoing process comprising the traits and physiological and morphological characteristics of acerola cherry (Malpighia emarginata DC.) variety Nutrilite Acerola Super C.

Another embodiment described herein is a cell from an acerola cherry (Malpighia emarginata DC.) seed or acerola cherry plant from the variety designated Nutrilite Acerola Super C. In one aspect, the cell comprises one or more cells or a tissue culture of cells from a Nutrilite Acerola Super C variety plant or seed. One aspect described herein is a tissue culture of cells produced from an acerola cherry Nutrilite Acerola Super C variety plant or plant part comprising an embryo, meristematic cell, leaf, cotyledon, hypocotyl, root, root tip, stem, pistil, anther, ovule, flower, pollen, or seed.

Another embodiment described herein is an acerola cherry plant regenerated from a cell or tissue culture derived from the Nutrilite Acerola Super C variety, where the plant comprises the morphological and physiological characteristics of variety Nutrilite Acerola Super C.

Another embodiment described herein is an acerola cherry plant or homogenous acerola cherry (Malpighia emarginata DC.) plant population comprising the Nutrilite Acerola Super C variety. Such acerola plant or plant population may be grown in a field, greenhouse, plant culture, aquaculture, or other means for growing plants. In one aspect, the population of Nutrilite Acerola Super C variety can be obtained by planting a plurality of Nutrilite Acerola Super C variety seeds in a suitable growth medium. In another aspect, the population of Nutrilite Acerola Super C variety may be obtained by grafting scion from one or more Nutrilite Acerola Super C plants onto appropriate rootstock and growing the grafted plants in a suitable growth medium. In one aspect, the rootstock is BRS 366 “Jaburu” or BRS 235 “Apodi.” Another aspect described herein is an acerola cherry plant produced by the foregoing methods comprising the traits and physiological and morphological characteristics of acerola cherry (Malpighia emarginata DC.) variety Nutrilite Acerola Super C.

Another embodiment described herein is one or more acerola cherry seeds harvested from a plant or homogeneous population of plants of the Nutrilite Acerola Super C variety.

Another embodiment described herein is a seed or homogenous population of acerola cherry (Malpighia emarginata DC.) seeds of the Nutrilite Acerola Super C variety comprising greater fruit yield, greater fruit mass, greater productivity, greater amounts of vitamin C, greater amounts of polyphenols, greater amounts of antioxidants, improved fruit firmness, greater juice yields, increased amounts of sugars, increased fruit uniformity, increased fruit yield per hectare, increased protein yield, increased fiber yield, increased fertilizer utilization, rapid growth, improved adaptation to winter planting, increased drought tolerance, increased cold tolerance, increased photoperiod, herbicide tolerance, increased insect resistance, increased disease resistance, or combinations thereof, as compared to other varieties of acerola plants.

Another embodiment described herein is a seed or homogenous population of acerola cherry (Malpighia emarginata DC.) seeds of the Nutrilite Acerola Super C variety comprising greater fruit yield, greater fruit mass, greater productivity, greater amounts of vitamin C, greater amounts of polyphenols, greater amounts of antioxidants, improved fruit firmness, greater juice yields, increased amounts of sugars, increased fruit uniformity, increased fruit yield per hectare, increased protein yield, increased fiber yield, increased fertilizer utilization, rapid growth, improved adaptation to winter planting, increased drought tolerance, increased cold tolerance, increased photoperiod, herbicide tolerance, increased insect resistance, increased disease resistance, or combinations thereof, as compared to other varieties of acerola plants.

Another embodiment described herein is a acerola cherry (Malpighia emarginata DC.) or homogenous population of acerola cherries (Malpighia emarginata DC.) plants of the Nutrilite Acerola Super C variety comprising greater fruit yield, greater fruit mass, greater productivity, greater amounts of vitamin C, greater amounts of polyphenols, greater amounts of antioxidants, improved fruit firmness, greater juice yields, increased amounts of sugars, increased fruit uniformity, increased fruit yield per hectare, increased protein yield, increased fiber yield, increased fertilizer utilization, rapid growth, improved adaptation to winter planting, increased drought tolerance, increased cold tolerance, increased photoperiod, herbicide tolerance, increased insect resistance, increased disease resistance, or combinations thereof, as compared to other varieties of acerola plants.

Another embodiment described herein is a homogenous population of acerola cherry (Malpighia emarginata DC.) plants of the Nutrilite Acerola Super C variety comprising greater fruit yield, greater fruit mass, greater productivity, greater amounts of vitamin C, greater amounts of polyphenols, greater amounts of antioxidants, improved fruit firmness, greater juice yields, increased amounts of sugars, increased fruit uniformity, increased fruit yield per hectare, increased protein yield, increased fiber yield, increased fertilizer utilization, rapid growth, improved adaptation to winter planting, increased drought tolerance, increased cold tolerance, increased photoperiod, herbicide tolerance, increased insect resistance, increased disease resistance, or combinations thereof, as compared to other varieties of acerola plants.

Another embodiment described herein is acerola cherry juice (Malpighia emarginata DC.) harvested of the Nutrilite Acerola Super C variety comprising greater fruit yield, greater fruit mass, greater productivity, greater amounts of vitamin C, greater amounts of polyphenols, greater amounts of antioxidants, improved fruit firmness, greater juice yields, increased amounts of sugars, increased fruit uniformity, increased fruit yield per hectare, increased protein yield, increased fiber yield, increased fertilizer utilization, rapid growth, improved adaptation to winter planting, increased drought tolerance, increased cold tolerance, increased photoperiod, herbicide tolerance, increased insect resistance, increased disease resistance, or combinations thereof, as compared to other varieties of acerola plants.

Another embodiment described herein is acerola cherry pulp (Malpighia emarginata DC.) harvested of the Nutrilite Acerola Super C variety comprising greater fruit yield, greater fruit mass, greater productivity, greater amounts of vitamin C, greater amounts of polyphenols, greater amounts of antioxidants, improved fruit firmness, greater juice yields, increased amounts of sugars, increased fruit uniformity, increased fruit yield per hectare, increased protein yield, increased fiber yield, increased fertilizer utilization, rapid growth, improved adaptation to winter planting, increased drought tolerance, increased cold tolerance, increased photoperiod, herbicide tolerance, increased insect resistance, increased disease resistance, or combinations thereof, as compared to other varieties of acerola plants.

Another embodiment described herein is a plant product produced from acerola cherry (Malpighia emarginata DC.) seed harvested from a plant or homogeneous plant population comprising the Nutrilite Acerola Super C variety. The plant product comprises one or more of acerola cherries, pulp, juice, extracts, vitamin C, polyphenols, antioxidants, sugars, protein, fiber, derivatives thereof, or combinations thereof. In one aspect, the plant product comprises acerola cherries, cherry juice, or cherry pulp. In one aspect, the plant product comprises juice. In one aspect, the juice has high concentrations of vitamin C, polyphenols, antioxidants, and sugars. Another aspect described herein is a food product, health supplement, or nutraceutical comprising acerola cherries, juice, pulp, extracts, derivatives thereof, or a combination thereof from the Nutrilite Acerola Super C variety. Another aspect is vitamin C, polyphenols, antioxidants obtained from the Nutrilite Acerola Super C variety. Another aspect described herein is acerola cherries, juice, or pulp from the Nutrilite Acerola Super C variety.

Another embodiment described herein is a crop population of plants of the Nutrilite Acerola Super C variety. In one aspect, the crop may be grown in a field, greenhouse, plant culture, hydroponic array, or other suitable growing area. In another aspect, the crop may be sown in one environment and then transplanted to another environment. For example, seeds may be sown and germinated in a greenhouse environment and then seedlings or immature plants transplanted to a field for growth until maturity or harvest. Alternatively, multiple grafted plants comprising Nutrilite Acerola Super C scion can be produced and transplanted to another growing area.

Another embodiment described herein is a plant part of an acerola cherry (Malpighia emarginata DC.) plant from the variety designated Nutrilite Acerola Super C. In one aspect, the plant part is the cherry fruit, fruit extract, juice, or juice extract.

Another embodiment described herein is a descendant of an acerola cherry (Malpighia emarginata DC.) seed or acerola plant from the variety designated Nutrilite Acerola Super C, the descendant comprising the traits and physiological and morphological characteristics of acerola cherry (Malpighia emarginata DC.) variety Nutrilite Acerola Super C. The Nutrilite Acerola Super C descendant may be a descendant plant or a seed of a descendant plant. In one aspect, the Nutrilite Acerola Super C descendant comprises one or more traits comprising greater fruit yield, greater fruit mass, greater productivity, greater amounts of vitamin C, greater amounts of polyphenols, greater amounts of antioxidants, improved fruit firmness, greater juice yields, increased amounts of sugars, increased fruit uniformity, increased fruit yield per hectare, increased protein yield, increased fiber yield, increased fertilizer utilization, rapid growth, improved adaptation to winter planting, increased drought tolerance, increased cold tolerance, increased photoperiod, herbicide tolerance, increased insect resistance, increased disease resistance, or combinations thereof, as compared to other varieties of acerola plants. In one aspect, the descendant is an F₁, F₂, F₃, F₄, F₅, F₆, F₇, F₈, F₉, F₁₀, or later generation of the Nutrilite Acerola Super C variety. In another embodiment, the descendant is a backcross descendant of the Nutrilite Acerola Super C variety. Another aspect described herein is a seed from a descendant from the variety designated Nutrilite Acerola Super C, comprising the traits and physiological and morphological characteristics of acerola cherry (Malpighia emarginata DC.) variety Nutrilite Acerola Super C.

Another embodiment described herein is germplasm of acerola cherry (Malpighia emarginata DC.) variety designated Nutrilite Acerola Super C. One aspect described herein is an acerola cherry plant generated from the Nutrilite Acerola Super C germplasm comprising the traits and physiological and morphological characteristics of acerola cherry (Malpighia emarginata DC.) variety Nutrilite Acerola Super C. In one aspect, a plant is generated from the germplasm and comprises one or more traits comprising greater fruit yield, greater fruit mass, greater productivity, greater amounts of vitamin C, greater amounts of polyphenols, greater amounts of antioxidants, improved fruit firmness, greater juice yields, increased amounts of sugars, increased fruit uniformity, increased fruit yield per hectare, increased protein yield, increased fiber yield, increased fertilizer utilization, rapid growth, improved adaptation to winter planting, increased drought tolerance, increased cold tolerance, increased photoperiod, herbicide tolerance, increased insect resistance, increased disease resistance, or combinations thereof, as compared to other varieties of acerola plants. In another aspect, one or more seeds are obtained from the germplasm-generated plant comprising the traits and physiological and morphological characteristics of the Nutrilite Acerola Super C variety.

Another embodiment described herein is a method for producing one or more Nutrilite Acerola Super C variety acerola cherries. In one aspect, the method for producing Nutrilite Acerola Super C seed comprises: (a) planting a seed of the Nutrilite Acerola Super C variety in pollinating proximity to itself; (b) growing a plant from the seeds planted in pollinating proximity; and harvesting one or more resultant cherries. Another aspect described herein is an acerola cherry produced by the foregoing method comprising the traits and physiological and morphological characteristics of acerola cherry (Malpighia emarginata DC.) variety Nutrilite Acerola Super C.

Another embodiment described herein is a method for producing one or more Nutrilite Acerola Super C variety acerola cherries. In one aspect, the method for producing Nutrilite Acerola Super C seed comprises: (a) grafting a scion of Nutrilite Acerola Super C variety to an appropriate rootstock; (b) growing the grafted plant; and harvesting one or more resultant cherries. In one aspect, the rootstock is BRS 366 “Jaburu” or BRS 235 “Apodi.” Another aspect described herein is an acerola cherry produced by the foregoing method comprising the traits and physiological and morphological characteristics of acerola cherry (Malpighia emarginata DC.) variety Nutrilite Acerola Super C.

Another embodiment described herein is a method for producing an acerola cherry (Malpighia emarginata DC.) variety plant by crossing a first parent acerola cherry (Malpighia emarginata DC.) plant with a second parent acerola (Malpighia emarginata DC.) plant where either the first or second parent acerola cherry plant is an acerola cherry plant of the line Nutrilite Acerola Super C. In another aspect, both the first and second parent acerola cherry (Malpighia emarginata DC.) plants can be of the variety Nutrilite Acerola Super C or descendants thereof. Any such methods using acerola cherry (Malpighia emarginata DC.) variety Nutrilite Acerola Super C may be employed including selfing, backcrosses, hybrid production, crosses to populations, and the like. This comprises all plants or seeds thereof produced using acerola cherry (Malpighia emarginata DC.) variety Nutrilite Acerola Super C as at least one parent, including those developed from varieties derived from or descendant from acerola cherry (Malpighia emarginata DC.) variety Nutrilite Acerola Super C. Advantageously, this acerola cherry (Malpighia emarginata DC.) variety can be used in crosses with other, different, acerola cherry (Malpighia emarginata DC.) plants to produce the first generation (F₁) acerola cherry (Malpighia emarginata DC.) hybrid seeds and plants with superior characteristics.

Another embodiment described herein is a method for producing a variety Nutrilite Acerola Super C-derived acerola cherry (Malpighia emarginata DC.) plant by crossing variety Nutrilite Acerola Super C with a second acerola cherry (Malpighia emarginata DC.) plant that comprises one or more agriculturally advantageous traits, obtaining seed from the resulting progeny; growing the progeny seed, and repeating the crossing and growing steps with the variety Nutrilite Acerola Super C-derived plant from 1 to 20 times. Another aspect described herein is the plants or seed produced from the foregoing methods or processes, comprising the traits and physiological and morphological characteristics of acerola cherry (Malpighia emarginata DC.) variety Nutrilite Acerola Super C and any other agriculturally advantageous traits from the second acerola cherry plant.

Another embodiment described herein is a method for producing one or more Nutrilite Acerola Super C variety acerola plants. In one aspect, the method for producing Nutrilite Acerola Super C seed comprises: (a) grafting a scion of Nutrilite Acerola Super C variety to an appropriate rootstock; (b) growing the grafted plant; and harvesting one or more resultant cherries. In one aspect, the rootstock is BRS 366 “Jaburu” or BRS 235 “Apodi.” Another aspect described herein is an acerola plant produced by the foregoing method comprising the traits and physiological and morphological characteristics of acerola cherry (Malpighia emarginata DC.) variety Nutrilite Acerola Super C.

Another embodiment described herein is a method for producing an acerola cherry (Malpighia emarginata DC.) variety Nutrilite Acerola Super C seed, the method comprising: (a) crossing a acerola cherry (Malpighia emarginata DC.) variety Nutrilite Acerola Super C plant with a second different acerola cherry (Malpighia emarginata DC.) variety Nutrilite Acerola Super C plant to produce F₁ acerola seed; and (b) harvesting the F₁ acerola cherry seed. Another aspect described herein is an acerola cherry seed produced by the foregoing method comprising the traits and physiological and morphological characteristics of acerola cherry (Malpighia emarginata DC.) variety Nutrilite Acerola Super C.

Another embodiment described herein is a method of using the variety Nutrilite Acerola Super C in selfing, backcrosses, hybrid production, crosses to populations, and similar procedures. Also described herein are plants or seeds produced using variety Nutrilite Acerola Super C as a parent, including plants descendant from or derived from variety Nutrilite Acerola Super C comprising the traits and physiological and morphological characteristics of acerola cherry (Malpighia emarginata DC.) variety Nutrilite Acerola Super C. Advantageously, the Nutrilite Acerola Super C variety is used in crosses with other, different, varieties to produce first generation (F₁) acerola cherry (Malpighia emarginata DC.) seeds and plants with superior agricultural characteristics.

Another embodiment described herein is a method of introducing a desired trait into acerola cherry (Malpighia emarginata DC.) variety Nutrilite Acerola Super C. In one aspect described herein, the method comprises: (a) crossing a Nutrilite Acerola Super C plant with a plant of another, different acerola cherry (Malpighia emarginata DC.) variety that comprises one or more agriculturally advantageous traits to produce F₁ progeny plants; (b) selecting one or more progeny plants that have the desired trait to produce selected progeny plants; (c) crossing the selected progeny plants with the Nutrilite Acerola Super C plants to produce backcross progeny plants; (d) selecting for backcross progeny plants that have the desired trait; and (e) repeating steps (c) and (d) three or more times in succession to produce fourth or higher backcross progeny plants that comprise the desired trait. Another aspect described herein is an acerola cherry plant or a seed from such plant produced by the foregoing method comprising the traits and physiological and morphological characteristics of acerola cherry (Malpighia emarginata DC.) variety Nutrilite Acerola Super C and one or more desired traits. The desired trait can comprise greater fruit yield, greater fruit mass, greater productivity, greater amounts of vitamin C, greater amounts of polyphenols, greater amounts of antioxidants, improved fruit firmness, greater juice yields, increased amounts of sugars, increased fruit uniformity, increased fruit yield per hectare, increased protein yield, increased fiber yield, increased fertilizer utilization, rapid growth, improved adaptation to winter planting, increased drought tolerance, increased cold tolerance, increased photoperiod, herbicide tolerance, increased insect resistance, increased disease resistance, or combinations thereof, as compared to other varieties of acerola plants.

Reproduction of the Nutrilite Acerola Super C variety can occur by natural processes, tissue culture, or regeneration. As used herein, the term “tissue culture” indicates a composition comprising isolated cells of the same or a different type or a collection of such cells organized into parts of a plant. Exemplary types of tissue cultures are protoplasts, calli, meristematic cells, and plant cells that can generate tissue culture. Means for preparing and maintaining plant tissue culture are well known in the art. Tissue culture comprising plant organs has been used to produce regenerated plants. See e.g., U.S. Pat. Nos. 5,959,185; 5,973,234; and 5,977,445, the disclosures of which are incorporated herein by reference.

Another embodiment described herein is one or more cells that can be grown and differentiated to produce acerola cherry (Malpighia emarginata DC.) plants having the physiological and morphological characteristics of variety Nutrilite Acerola Super C. Another aspect described herein is acerola cherries produced from such tissue-culture derived plants having the physiological and morphological characteristics of variety Nutrilite Acerola Super C. Other aspects described herein are acerola plants derived from or descendant from tissue-culture derived plants having the physiological and morphological characteristics of variety Nutrilite Acerola Super C.

Another embodiment described herein is the transformation of the Nutrilite Acerola Super C variety with exogenous genes to impart new or enhanced agriculturally advantageous traits using protocols known to those of skill in the art. Another aspect described herein is the culture, plants, or seed produced from the transformation, comprising the traits and physiological and morphological characteristics of variety Nutrilite Acerola Super C and any other agriculturally advantageous traits.

Another embodiment described herein is a method for developing novel acerola cherry (Malpighia emarginata DC.) plants or seeds based on the Nutrilite Acerola Super C variety. In one embodiment, the specific type of breeding method is pedigree selection, where both single plant selection and mass selection practices are employed. Pedigree selection is described by Walter R. Fehr, Principles of Cultivar Development, Macmillan Pub. Co. (1993), which is incorporated by reference herein for such teachings.

Acerola cherry (Malpighia emarginata DC.) plants may be selected for agriculturally advantageous characteristics. In one embodiment, the pedigree method of breeding is practiced where selection is first practiced among F₂ plants. In the next season, the most desirable F₃ lines are first identified, and then desirable F₃ plants within each line are selected. The following season and in all subsequent generations of inbreeding, the most desirable families are identified first, then desirable lines within the selected families are chosen, and finally desirable plants within selected lines are harvested individually. A family refers to lines that were derived from plants selected from the same progeny row as the preceding generation.

Using the pedigree method, two parents may be crossed using an emasculated female and a pollen donor (male) to produce F₁ offspring or open pollination. Methods of removing pollen, such as misting to wash the pollen off prior to fertilization, may be employed to assure crossing or hybridization. The F₁ may be self-pollinated to produce a segregating F₂ generation. Individual plants may then be selected which represent the desired phenotype in each generation (F₃, F₄, F₅, etc.) until the traits are homozygous or fixed within a breeding population.

In addition to crossing, selection may be used to identify and isolate new acerola cherry (Malpighia emarginata DC.) lines comprising agriculturally advantageous characteristics. In acerola cherry (Malpighia emarginata DC.) selection, acerola seeds are planted, the plants are grown, and single plant selections are made of plants with desired characteristics. Seed from the single plant selections may be harvested, separated from seeds of the other plants in the field, and re-planted. The plants from the selected seed may be monitored to determine whether they exhibit the desired characteristics of the originally selected line. Selection work is preferably continued over multiple generations to increase the uniformity of the new line.

The choice of a breeding or selection method depends on the mode of plant reproduction, the heritability of the trait(s) being improved, and the type of variety used commercially (e.g., F₁ hybrid variety, pure line variety, etc.). For highly heritable traits, the evaluation of superior individual plants evaluated at a single location will be effective; whereas for traits with low heritability, selection should be based on mean values obtained from replicated evaluations of families of related plants, typically grown at different locations to normalize for environmental factors. Popular selection methods commonly include pedigree selection, modified pedigree selection, mass selection, and recurrent selection.

The complexity of inheritance influences choice of the breeding method. Backcross breeding may be used to transfer one or more agriculturally advantageous traits into a desirable variety. This approach has been used extensively for breeding disease-resistant varieties. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program may include a periodic, objective evaluation of the efficiency of the breeding procedure. Evaluation criteria may vary depending on the goal and objectives, but should include gain from selection per year based on comparisons to an appropriate standard, the overall value of the advanced breeding lines, and the number of successful varieties produced per unit of input (e.g., per year, per dollar expended, etc.).

In another embodiment, promising advanced breeding lines are thoroughly tested and compared to appropriate standards in environments representative of the commercial target area(s). The best lines are candidates for new commercial varieties; those deficient in a few traits can be used as parents to produce new populations for further selection.

These processes, which lead to the final step of marketing and distribution, usually take several years from the time the first cross or selection is made. Therefore, development of new varieties is a time-consuming process that requires precise forward planning, efficient use of resources, and adherence to the planned process.

The identification of individual plants that are genetically superior can be difficult, because for most traits, the true genotypic value may be masked by other confounding plant traits or environmental factors. One method for identifying superior plants is to observe their performance relative to other experimental plants and to a widely grown standard variety. If a single observation is inconclusive, replicated observations provide a better evaluation of the genetic worth.

The goal of acerola cherry (Malpighia emarginata DC.) plant breeding is to develop new, unique, and superior acerola cherry (Malpighia emarginata DC.) varieties having one or more agriculturally advantageous characteristics. In one embodiment, two or more parental lines are crossed, followed by repeated selfing, open pollination, and selection, producing many new genetic combinations. Millions of different genetic combinations can be generated via crossing, selfing, and mutations. Each year germplasm is selected to advance to the next generation. This germplasm may be grown under different geographical, climatic, and soil conditions, and further selections are then made during and at the end of the growing season.

In another embodiment, the development of commercial acerola cherry (Malpighia emarginata DC.) varieties requires the development of acerola cherry (Malpighia emarginata DC.) varieties, the crossing of these varieties, and the evaluation of the crosses for agriculturally advantageous characteristics. Pedigree breeding and recurrent selection breeding methods may be used to develop varieties from breeding populations. Breeding programs may combine desirable traits from two or more varieties or various broad-based sources into breeding pools from which varieties are developed by selfing and selection of desired phenotypes. The new varieties may be crossed with other varieties and the hybrids from these crosses are evaluated to determine which have commercial potential.

Another embodiment described herein is a method for developing variety Nutrilite Acerola Super C progeny acerola cherry (Malpighia emarginata DC.) plants in a acerola plant breeding program comprising: obtaining an acerola plant, or a part thereof, of variety Nutrilite Acerola Super C, utilizing the plant or plant part as a source of breeding material, and selecting an acerola variety Nutrilite Acerola Super C progeny plant with molecular markers in common with variety Nutrilite Acerola Super C and/or with morphological and/or physiological characteristics listed in Table 7 or described herein. Such characteristics can comprise one or more of increased greater fruit yield, greater fruit mass, greater productivity, greater amounts of vitamin C, greater amounts of polyphenols, greater amounts of antioxidants, improved fruit firmness, greater juice yields, increased amounts of sugars, increased fruit uniformity, increased fruit yield per hectare, increased protein yield, increased fiber yield, increased fertilizer utilization, rapid growth, improved adaptation to winter planting, increased drought tolerance, increased cold tolerance, increased photoperiod, herbicide tolerance, increased insect resistance, increased disease resistance, or combinations thereof, as compared to other varieties of acerola plants. Breeding steps that may be used in the acerola cherry (Malpighia emarginata DC.) plant breeding program include pedigree breeding, backcrossing, mutation breeding, and recurrent selection. In conjunction with these steps, techniques such as RAPD-enhanced selection, genetic marker enhanced selection (for example SSR markers) and the making of double haploids may be utilized.

Another embodiment described herein is a method producing a population of acerola cherry (Malpighia emarginata DC.) variety Nutrilite Acerola Super C-progeny acerola cherry (Malpighia emarginata DC.) plants, comprising crossing variety Nutrilite Acerola Super C with another acerola cherry (Malpighia emarginata DC.) plant, thereby producing a population of acerola cherry (Malpighia emarginata DC.) plants, which, on average, derive 50% of their alleles from acerola cherry (Malpighia emarginata DC.) variety Nutrilite Acerola Super C. A plant of this population may be selected and repeatedly selfed or sibbed with an acerola cherry (Malpighia emarginata DC.) variety resulting from these successive filial generations. One embodiment is the acerola cherry (Malpighia emarginata DC.) variety produced by the method that has obtained at least 50% of its alleles from acerola cherry (Malpighia emarginata DC.) variety Nutrilite Acerola Super C and having the physiological and morphological characteristics of variety Nutrilite Acerola Super C.

Another embodiment described herein is acerola cherry (Malpighia emarginata DC.) variety Nutrilite Acerola Super C progeny plants comprising a combination of one or more variety Nutrilite Acerola Super C traits comprising any of those listed in Table 7 or described herein, so that the progeny acerola plant is not significantly different for the traits than acerola cherry (Malpighia emarginata DC.) variety Nutrilite Acerola Super C as determined at the 5% significance level when grown in the same environmental conditions. Using techniques described herein, molecular markers may be used to identify the progeny plant as an acerola cherry (Malpighia emarginata DC.) variety Nutrilite Acerola Super C progeny plant. Mean trait values may be used to determine whether trait differences are significant, and preferably, the traits are measured on plants grown under the same environmental conditions. Once such a variety is developed its value is substantial since it is important to advance the germplasm base as a whole in order to maintain or improve traits such as yield, disease resistance, pest resistance, and plant performance in extreme environmental conditions.

Progeny of acerola cherry (Malpighia emarginata DC.) variety Nutrilite Acerola Super C may also be characterized through their filial relationship with acerola cherry (Malpighia emarginata DC.) variety Nutrilite Acerola Super C, as for example, being within a certain number of breeding crosses of acerola cherry (Malpighia emarginata DC.) variety Nutrilite Acerola Super C. A breeding cross is a cross intended to introduce new genetics into the progeny, and is distinguished from a cross, such as a self or a sib cross, made to select among existing genetic alleles. The lower the number of breeding crosses in the pedigree, the closer the relationship between acerola cherry (Malpighia emarginata DC.) variety Nutrilite Acerola Super C and its progeny. Progeny produced by the methods described herein may be within 1, 2, 3, 4, or 5 crosses of acerola cherry (Malpighia emarginata DC.) variety Nutrilite Acerola Super C.

Breeding methods that are used for selecting agriculturally advantageous characteristics or traits in crops are known in the art. See Robert W. Allard, Principles of Plant Breeding, 2^{nd} ed. John Wiley and Son, (2010), which is incorporated by reference herein for such teachings. The following breeding methods may be used with acerola cherry (Malpighia emarginata DC.) variety Nutrilite Acerola Super C in the development of further acerola cherry (Malpighia emarginata DC.) plants.

Pedigree breeding is used commonly for the improvement of self-pollinating crops or inbred lines of cross-pollinating crops. Two parents comprising favorable, complementary traits are crossed to produce an F₁. An F₂ population is produced by selfing one or several F₁s or by intercrossing two F₁s (sibling mating). Selection of the best individuals typically occurs in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (e.g., F₆ and F₇), the best lines, or mixtures of phenotypically similar lines are tested for potential release as new varieties.

Mass and recurrent selections can be used to improve populations of either self- or cross-pollinating crops. A genetically variable population of heterozygous individuals may be identified or created by intercrossing several different parents. The best plants may be selected based on individual superiority, outstanding progeny, or excellent combining ability. Preferably, the selected plants are intercrossed to produce a new population comprising agriculturally advantageous characteristics in which further cycles of selection are continued.

Backcross breeding has been used to transfer genes for a simply inherited, highly heritable trait into a desirable homozygous variety or line that is the recurrent parent. The source of the trait to be transferred is called the donor parent. The resulting plant is expected to have the attributes of the recurrent parent (e.g., variety) and the desirable trait transferred from the donor parent. After the initial cross, individuals possessing the phenotype of the donor parent may be selected and repeatedly crossed (backcrossed) to the recurrent parent. The resulting plant is expected to have the attributes of the parent and the desirable traits transferred from the donor parent.

The single-seed descent procedure refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advancement is completed.

In a multiple-seed procedure, one or more seeds from each plant in a population is harvested and combined together to form a bulk. Part of the bulk is used to plant the next generation and part is put in reserve. The procedure has been referred to as modified single-seed descent or the pod-bulk technique. The multiple-seed procedure conserves time and labor resources at harvest. It is considerably faster to obtain bulk seed than to select single seeds from each by hand. The multiple-seed procedure facilitates planting the same number of seeds from a population for each generation of inbreeding. Enough seeds are harvested to compensate for plants that did not germinate or produce seed.

Mutation breeding is a method for introducing new agriculturally advantageous characteristic into acerola cherry (Malpighia emarginata DC.) varieties. Spontaneous mutations or those artificially induced are useful sources of variability. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneimines, sulfates, sulfonates, sulfones, or lactones), azide, hydroxylamine, nitrous acid, acridines, or other mutagens. Once a desired trait is observed through mutagenesis the trait may then be incorporated into existing germplasm by traditional breeding techniques. Protocols for mutation breeding can be found in Walter R. Fehr, Principles of Cultivar Development, Macmillan Pub. Co. (1993), which is incorporated by reference for such teachings.

Another embodiment described herein is the mutagenesis of the Nutrilite Acerola Super C variety with chemical mutagens or ionizing radiation to induce one or more mutations and then selecting for one or more agriculturally advantageous traits by methods known in the art. For example, seeds of Nutrilite Acerola Super C variety could be treated with chemical mutagens, planted, and then the resulting plants selected for an agriculturally advantageous trait, such as herbicide tolerance, cold tolerance, drought tolerance, or insect resistance.

The production of double haploids can also be used for the development of homozygous varieties in a breeding program. Double haploids are produced by the doubling of a set of chromosomes from a heterozygous plant to produce a completely homozygous individual. See Wan et al., “Efficient production of doubled haploid plants through colchicine treatment of anther-derived maize callus,” Theor. Appl. Genet., 77:889-892 (1989), which is incorporated by reference.

### Additional Embodiments

One embodiment relates to a method for producing an acerola cherry, the method comprising:
(a) planting a seed of acerola cherry (Malpighia emarginata DC.) variety designated Nutrilite Acerola Super C, a representative sample of seed having been deposited under ATCC Patent Deposit Designation, Patent Deposit No. PTA-126522, in pollinating proximity to itself or to a seed from a different acerola variety;
(b) growing a plant from the seeds planted in pollinating proximity; and
(c) harvesting one or more resultant acerola cherries.

In the method described immediately above, the acerola cherry comprises one or more of the characteristics:
fruit firmness of about 8 to 12 N;
juice yield of about 50-70%;
vitamin C content of about 3000-3500 mg·100 g⁻¹;
total soluble phenols of about 4000-4500 mg GAE·100 g⁻¹; or
total antioxidant activity of about 300-350 μmol TEAC·g⁻¹ pulp.

Another embodiment relates to a method for producing an acerola cherry, the method comprising:
(a) grafting a scion of an acerola cherry plant (Malpighia emarginata DC.) variety designated Nutrilite Acerola Super C, a representative sample of seed having been deposited under ATCC Patent Deposit Designation, Patent Deposit No. PTA-126522, onto an acerola rootstock;
(b) growing the grafted plant; and
(c) harvesting one or more resultant acerola cherries.

In the method described immediately below, the acerola cherry comprises one or more of the characteristics:
fruit firmness of about 8 to 12 N;
juice yield of about 50-70%;
vitamin C content of about 3000-3500 mg·100 g⁻¹;
total soluble phenols of about 4000-4500 mg GAE·100 g⁻¹; or
total antioxidant activity of about 300-350 μmol TEAC·g⁻¹ pulp.

In the method described immediately above, the rootstock comprises acerola variety BRS 366 “Jaburu” or BRS 235 “Apodi.”

Yet, another embodiments relates to a method for producing acerola cherries (Malpighia emarginata DC.) variety Nutrilite Acerola Super C, the method comprising:
(a) crossing an acerola cherry (Malpighia emarginata DC.) variety designated Nutrilite Acerola Super C, a representative sample of seed having been deposited under ATCC Patent Deposit Designation, Patent Deposit No. PTA-126522, with a plant of a second, different acerola cherry (Malpighia emarginata DC.) variety to produce F₁ acerola cherries; and
(b) harvesting the F₁ acerola cherries.

An additional embodiment relates to an F₁ acerola cherry (Malpighia emarginata DC.) produced by the method described immediately above.

Yet further embodiment relates to a method of introducing a desired trait into acerola cherry (Malpighia emarginata DC.) variety Nutrilite Acerola Super C, the method comprising:
(a) crossing acerola cherry (Malpighia emarginata DC.) variety designated Nutrilite Acerola Super C, a representative sample of seed having been deposited under ATCC Patent Deposit Designation, Patent Deposit No. PTA-126522, with a plant of another acerola cherry (Malpighia emarginata DC.) variety that comprises a desired trait to produce F₁ progeny plants;
(b) selecting one or more progeny plants that have the desired trait to produce selected progeny plants;
(c) crossing the selected progeny plants with the Nutrilite Acerola Super C plants to produce backcross progeny plants;
(d) selecting for backcross progeny plants that have the desired trait; and
(e) repeating steps (c) and (d) three or more times in succession to produce fourth or higher backcross progeny plants that comprise the desired trait.

One further embodiments relates to the acerola cherry (Malpighia emarginata DC.) plant produced by the method described immediately above, wherein the plant comprises traits and physiological and morphological characteristics of acerola cherry (Malpighia emarginata DC.) variety Nutrilite Acerola Super C, a representative sample of seed having been deposited under ATCC Patent Deposit Designation, Patent Deposit No. PTA-126522.

Yet further embodiment relates to the acerola cherry (Malpighia emarginata DC.) plant as described immediately above, wherein the desired trait comprises one or more of: greater fruit yield, greater fruit mass, greater productivity, greater amounts of vitamin C, greater amounts of polyphenols, greater amounts of antioxidants, improved fruit firmness, greater juice yields, increased protein yield, increased fiber yield, increased fertilizer utilization, improved adaptation to winter planting, increased drought tolerance, increased cold tolerance, increased photoperiod, herbicide tolerance, increased insect resistance, increased disease resistance, or combinations thereof, as compared to other varieties of acerola plants or wild-type acerola.

One additional embodiment relates to a method for selecting an acerola cherry (Malpighia emarginata DC.) plant comprising the traits and the physiological and morphological characteristics of acerola cherry (Malpighia emarginata DC.) variety Nutrilite Acerola Super C, a representative sample of seed having been deposited under ATCC Patent Deposit Designation, Patent Deposit No. PTA-126522, the method comprising:
performing one or more cross pollination selections; and
performing one or more vegetative propagations and selections;

wherein the selection selects for one or more of greater fruit yield, greater fruit mass, greater productivity, greater amounts of vitamin C, greater amounts of polyphenols, greater amounts of antioxidants, improved fruit firmness, greater juice yields, increased protein yield, increased fiber yield, increased fertilizer utilization, improved adaptation to winter planting, increased drought tolerance, increased cold tolerance, increased photoperiod, herbicide tolerance, increased insect resistance, increased disease resistance, or combinations thereof, as compared to other varieties of acerola plants or wild-type acerola.

It will be readily apparent to one of ordinary skill in the relevant arts that suitable modifications and adaptations to the compositions, methods, and applications described herein can be made without departing from the scope of any embodiments or aspects thereof. The plant varieties, seeds, compositions, processes, and methods provided are exemplary and are not intended to limit the scope of any of the specified embodiments. All of the various embodiments, aspects, and options disclosed herein can be combined in any variations or iterations. The scope of all plant varieties, seeds, hybrids, crosses, compositions, formulations, methods, and processes described herein include all actual or potential combinations of embodiments, aspects, options, examples, and preferences herein described. All patents, publications, and non-patent literature cited herein are incorporated by reference herein for the specific teachings thereof. The citation of any references herein is not an admission that such references are prior art. If any material incorporated by reference contradicts or is inconsistent with this specification, the specification will supersede any such material.

### REFERENCES

Ainsworth, EA, Gillespie, KM, “Estimation of total phenolic content and other oxidation substrates in plant tissues using Folin-Ciocalteu reagent,” Nature Protocols 2: 875-877 (2007); doi.org/10.1038/nprot.2007.102.
Almeida, JPN, Dantas, LLGR, Arrais, IG, Tosta, MS, Mendonça, V, “Fungo micorrízico arbuscular e extrato de algas no crescimeto inicial de portaenxerto de aceroleira,” Revista de Ciências Agrárias, Fortaleza 57(1): 22-28 (2014); dx.doi.org/10.4322/rca.2013.061.
Asad, HA, Meah, MB, Begum, SN, Khalil, MI, Rafii, MY, Latif, MA, “Study of genetic variation of eggplant cultivars by using RAPD-PCR molecular markers and the relationship with Phomopsis blight disease reaction,” Genetics and Molecular Research, (2015); doi: 10.4238/2015.December.16.1.
Brasil Ministerio da Agricultura, Pecuária e Abastecimento, “Registro Nacional de Cultivares,” 2018 Disponível em: sistemas.agricultura.gov.br/snpc/cultivarweb/cultivares_registradas.php.
Carpentieri-Pípolo, V, Destro, D, Prete, CEC, Gonzales, MGN, Popper, I, Zanatta, S, Silva, FAM, “Seleção de genótipos parentais de acerola com base na divergência genética multivariada,” Pesq. Agropec. Brasília 35(8): 1613-1619 (2000).
Dias, FTC, Bertini, CHCM, Silva, APM, Cavalcanti, JJV, “Variabilidade genética de feijão-caupi de porte ereto e ciclo precoce analisada por marcadores RAPD e ISSR,” Revista Ciência Agronômica, 46(3): 563-572 (2015); doi: 10.5935/1806-6690.20150039.
Fachi, LR, Ferreira, AFN, Gaburgio, E LS, Krause, W, “Qualidade e correlação dos parâmetros físico-químicos dos frutos de cultivares de acerola,” Enciclopédia biosfera, Centro Científico Conhecer-Goiânia, 13(24): 891 (2016).
Franca, LGDA, “Indicação de clones de aceroleira visando a qualidade de frutos verdes para processamento,” Limoeiro do Norte-CE, Dissertação (Mestrado em Tecnologia de Alimentos)—Instituto Federal de Educação, Ciência e Tecnologia do Ceará, Campus Limoieiro do Norte, 2016.
Freire, V, “Acerola: Embrapa e Nutrilite lançam nova cultivar mais produtiva,” Cultivar Acerola BRS 366 Jaburu. Fortaleza, 2012. Disponível em: www.gestaonocampo.com.br/biblioteca/acerola-embrapa-e-Nutrilite lancam-novacultivar mais-produtiva/ Acesso em: 18/2/2017.
Furlaneto, FPB, Nasser, MD, “Panorama da cultura da acerola no estado de São Paulo,” Pesquisa e Tecnologia 12(1): 1-6 (2015).
Hammer, DAT, Harper, Ryan, PD., “PAST: Paleontological statistics software package for education and data analysis,” Palaeontologia Electronica (2001) Disponível em: palaeo-electronica.org/2001_1/past/issue1_01.htm.
Johnson, PD, Acerola (Malpighia glabra L., M. punicifolia L., M. emarginata D.C.): agriculture, production and nutrition, World Rev. Nutr. Diet. 91:67-75 (2003); doi.org/10.1159/000069930.
Larrauri, JA, Pupérez, P, Saura-calixto, F. “Effect of drying temperature on the stability of polyphenols and antioxidant activity of red grape pomace peels,” J. Agricultural and Food Chemistry 45:1390-1393 (1997); doi: 10.1021/jf960282f.
Lima, EM, Araujo, MEB, Bertini, CHCM, Moura, CFH, Hawerroth, MC, “Diversidade genética de clones de aceroleira avaliada por meio de marcadores moleculares ISSR,” Com. Sci., Bom Jsus, 6(2): 174-180 (2015).
Martins, EA, Campos, RT, Campos, KC, Almeida, CS, “Rentabilidade da Produção de Acerola Orgânica Sob Condição Determinística e de Risco: estudo do distrito de irrigação Tabuleiro Litorâneo do Piauí,” Revista de Economia e Sociologia Rural 54(1):9-28 (2016); dx.doi.org/10.1590/1234-56781806-9479005401001.
Mondin, MA, de Oliveira, CA, Vieira MLC, “Karyotype characterization of Malpighia emarginata (Malpighiaceae),” Rev. Bras. Frutic. 32(2) (2010); doi.org/10.1590/S0100-29452010005000072.
Moraes Filho, RM, Martins, LSS, Musser, RS, Montarroyos, AVV, Silva, EF, “Genetic variability in accessions of the acerola germplasm bank of Universidade Federal Rural de Pernambuco, Brazil,” Genetic Molecular Research (2013); doi: 10.4238/2013.October.29.8.
Mortin, JF, “Barbados Cherry,” in Fruits of Warm Climates, 204-207 (1987), Miami FL. Available at: hort.purdue.edu/newcrop/morton/barbados_cherry.html.
Moura, FH, Alves, RE, Figueiredo, RW, Paiva, JR, “Avaliações físicas e físico-químicas de frutos de clones de aceroleira (Malpighia emarginata DC.),” Rev. Ciênc. Agron., 38(1): 52-57 (2007); ISSN 0045-6888.
Paiva, JR, “Cultivares e melhoramento genético,” In: Manica, I, Icuma, IM, Fioravanço, JC, Paiva, JR, Paiva, MC, Junqueira, NTV, “Acerola: tecnologia de produção, pós-colheita, congelamento, exportação, mercados,” Porto Alegre: Cinco Continentes, 69-88 (2003).
Pontes, ATAC, Soares, FAX, Lima, FWM, Diniz, CV, “Uso do ciclo fenológico da aceroleira para padronização do ponto de colheita mecanizada,” In: Congresso Brasileiro de Engenharia Agrícola-CONBEA, São Pedro-SP. Jubileu de ouro do SBEA (2015); ISSN: 2358-582X.
Righetto, AM, “Caracterização físico-química e estabilidade de suco de acerola verde microencapsulado por atomização e liofilização/Tese (Doutorado)” Universidade Estadual de Campinas. Faculdade de Engenharia de Alimentos. Campinas, 2003.
Salla, MFS, Ruas, CDF, Ruas, PM, Carpentieri-Pípolo, EV, “Uso de marcadores moleculares na análise da variabilidade genética em acerola (Malpighia emarginata DC.),” Rev. Bras. Frutic., 24(1):15-22 (2002); dx.doi.org/10.1590/S0100-29452002000100005.
Souza, KO, Moura, CFH, Brito, ES, Miranda, MRA, “Antioxidant compounds and total antioxidant activity in fruits of acerola from cv. Flor Branca, Florida Sweet and BRS 366,” Revista Brasileira de Fruticultura 36(2):294-304 (2014); dx.doi.org/10.1590/0100-2945-410/13.
Souza, KO, Moura, CFH, Lopes, MMA, Rabelo, MC, Miranda, MRA, “Qualidade pós-colheita de acerolas (M. emarginata) tratadas com ácido giberélico e armazenadas sob refrigeração,” Rev. Brasileira de Fruticultura 39 (2017); dx.doi.org/10.1590/0100-29452017574.
Strohecker, R, Henning, HM, “Análises de vitaminas: métodos comprovados,” Madrid: Paz Montolvo, 428, 1967.
Viana, JPG, Pires, CJ, Pinheiro, JB, Valente, SES, Lopes, ACA, Gomes, RLF, “Genetic diversity in garlic germplasm,” Ciência Rural, Santa Maria, 2015; dx.doi.org/10.1590/0103-8478cr20130988.

### EXAMPLES

### Example 1

### Plant Materials and Crop Conditions

The experimental material was obtained from the germplasm bank of the Amway Nutrilite Brazil Farm, Ubajara, Ceará, Brazil, located at latitude South 3° 51′ 12″, and longitude West 41° 5′ 10″ at an altitude of 710 m. The mean annual temperature is 25° C. and the mean annual rainfall is 600 mm. Agricultural conditions were organic and the main fertilizers used were organic compost and castor bean cake as a nitrogen source.

The acerola genotypes were planted in plots under the same soil conditions, cultivation practices, and irrigation systems and were spaced at intervals of 1.5×3.0 m. The new genotypes were propagated by seeds and subsequently multiplied asexually.

### Initial Matrices and First Selection

The study was initiated with matrice seeds from 20 different acerola genotypes (varieties). Over a 3-year period, these genotypes grew into trees and were allowed to cross-pollinate freely through natural pollination (approximately 7 flowering cycles per year). There was a high genetic divergence in the acerola plants that cross-pollinated over this three-year period.

Based on fruit yields and vitamin C yields obtained from the 20 three-year old genotypes, the 10 most productive genotypes (e.g., highest fruit yield and greatest vitamin C content) were selected and seeds were obtained. Seeds were harvested from tree-year old plants during the rainy season (January to April) over about 3 flowering cycles.

### Second Selection

Progeny plants (F₂) grown from seeds of the 10 most productive genotypes were grown and allowed to pollinate through natural pollination. The desirable agronomic characteristics were evaluated over a two-year period. The six most productive genotypes (highest fruit yield and vitamin C content) were identified (e.g., genotypes A-F). These genotypes were propagated by grafting.

Grafting was performed by transferring branches from the genotypes on to one of two rootstock varieties, BRS 366 “Jaburu” and BRS 235 “Apodi.” The grafting method is described below.

### Grafting Method

Desirable acerola genotypes were propagated by whip grafting single branches from the desired genotype scion onto multiple rootstock plants. This permits the rapid production of clonal populations of the genotypes. The grafting method involved selecting and cutting branches from the genotypes that are approximately 20 cm long, 0.3 to 0.5 mm diameter, and having at least 5 nodes. Immature or overly mature branches or branches with pests or diseases were not used. The cut branches were placed in a cooler within 40 min of cutting to prevent dehydration; the cut branches may be stored in the cooler for up to 18 hours.

Rootstock plants were produced from seed using varieties with excellent germination rates. Typical varieties used for rootstock are BRS 366 “Jaburu” and BRS 235 “Apodi.” Plants of about 6 months post germination were used for rootstock.

Tools for grafting were sanitized with 70% ethanol. The graftings were conducted in a shade house (50% shade). A rootstock plant with a shoot diameter similar to the grafted branch was selected; the leaves were removed; and the rootstock shoot was cut below the nodule (about 8 cm up). The cut rootstock was slit longitudinally about 2.5 cm into the shoot top towards the root, creating a V-notch. The genotype scion branch for grafting was prepared by removing 0.5 cm from the bottom (to remove any dehydrated tissue) and then slicing off a 2.5 cm diagonal section on each side of the branch that will mate with the rootstock notch in a V-like tongue and grove manner. The scion and rootstock were joined with the exposed tissue adjacent to one another. Biodegradable tape was wrapped around the graft juncture to secure it. The top of the scion was removed, leaving 4 to 5 nodes. The grafted shoots were then covered with individual plastic bags.

Then newly grafted plants with the shoots bagged were transferred to an adaptation shade house (75% shade). After 15 to 30 days, depending on leaf growth, the plastic bags were removed. The bags were removed after sunset to avoid leaf damage followed by sun exposure. Grafted plants were maintained in the 75% shade house for approximately 90 days from the date of grafting. After adaptation, the grafted plants were transferred to fields (clonal gardens). The total process post-grafting to field planting is about 6-8 months.

### Third Selection

The six experimental genotypes identified in Selection 2 were named Genotypes A-F. In addition to these six genotypes, four cultivars commonly grown in the region were used as reference varieties, e.g., BRS 366 “Jaburu,” BRS 235 “Apodi,” Flor Branca, and Monami. The experimental design was a randomized block with 10 genotypes and six clonal replications, totaling 60 plants. The six genotypes (A-F) and the four common cultivars were evaluated over two years for agronomic, morphological, phytochemical, and molecular characteristics. Genotype A was selected as the most productive variety and named Nutrilite Acerola Super C.

### Evaluation of Genotypes

The phenotypic characterization (morphology, yield, agronomic characteristics) was based on the agronomic descriptors table from the Brazilian Ministry of Agriculture and was applied to trees at the third production year. The productivity was evaluated for two years and the fruits were harvested and measured at each growing cycle. Immature fruits were used for the physical characteristics determination such as weight, volume, diameter, and firmness. Subsequently, the samples were frozen and then analyzed for vitamin C content, total polyphenols, antioxidant capacity, sugars, and pH. The analyses were performed using composite samples with four replicates per clonal group. The results were evaluated by analysis of variance (ANOVA) and the means were compared by the Tukey test (p>0.05), using the software MINITAB 16. A descriptive analysis was performed for the agronomic variables to verify distinctions between the progenies and the commercial varieties. The acerola genotypes were characterized using random amplified polymorphic DNA (RAPD) in order to obtain the genetic distance between the genotypes. These analyses are described in the following examples.

### Example 2

### Productivity

The acerola fruits were harvested from the grafted genotypes and weighed separately at each production cycle, before complete physiological maturation, at the P5 stage, using the acerola fruits classified according the maturation stage—P1 to P7 (Pontes et al., 2015). The goal was to obtain the greatest vitamin C content and productivity. The evaluations were carried out for two years, 2016 and 2017, and the results expressed in kg·tree⁻¹.

Productivity, fruit mass, fruit firmness, juice yield, vitamin C content, total polyphenols and total antioxidant activity were compared among genotypes using analysis of variance (ANOVA) and the means compared by the Tukey test (p<0.05) using the statistical program MINITAB, version 16 (Minitab Inc., State College, PA, USA).

Table 1 shows productivity results for all studied genotypes. Genotypes A (Nutrilite Acerola Super C) and Monami were the most productive in both years.

TABLE 1
Analysis of Variance of Acerola Genotype Productivity
(Ubajara, Ceará 2016 and 2017)

| | Productivity (kg · tree⁻¹) | | | |
|---|---|---|---|---|
| | 2016 | | 2017 | |
| Genotype | Mean | CV (%) | Mean | CV (%) |
| A* | 28.86 ^{a} | 14.1 | 50.6 ^{a} | 4.01 |
| B | 18.10 ^{c} | 19.5 | 33.2 ^{c} | 4.90 |
| C | 11.54 ^{e} | 13.6 | 31.4 ^{d} | 3.75 |
| D | 15.01 ^{d} | 16.32 | 32.8 ^{c} | 18.3 |
| E | 13.95 ^{d} | 15.77 | 35.2 ^{c} | 4.94 |
| F | 11.24 ^{e} | 17.71 | 30.0 ^{d} | 6.36 |
| BRS 235 | 21.26 ^{e} | 5.07 | 41.1 ^{b} | 3.09 |
| BRS 366 | 21.17 ^{b} | 1.90 | 37.3 ^{c} | 8.09 |
| Flor Branca | 22.41 ^{b} | 4.23 | 41.6 ^{b} | 3.63 |
| Monami | 28.18 ^{a} | 4.34 | 51.4 ^{a} | 5.52 |
| Average | 19.17 | | 38.46 | |

*Nutrilite Acerola Super C
Different letters on columns indicate significant differences by the Tukey's test (p < 0.05).

Table 2 shows the mean fruit mass for the genotypes.

TABLE 2
Analysis of Variance of Acerola Genotype Fruit Mass
(Ubajara, Ceará 2016 and 2017)

| | Mass of fruit (g) | | |
|---|---|---|---|
| Genotype | Mean | SD | CV (%) |
| A* | 4.38 ^{a} | ±0.23 | 5.4 |
| B | 3.90 ^{cd} | ±0.91 | 23.4 |
| C | 3.97 ^{c} | ±0.11 | 2.9 |
| D | 3.56 ^{d} | ±0.2 | 5.8 |
| E | 3.92 ^{cd} | ±0.13 | 3.5 |
| F | 5.03 ^{a} | ±0.08 | 1.6 |
| BRS 235 | 5.07 ^{a} | ±0.45 | 8.9 |
| BRS 366 | 4.80 ^{a} | ±0.86 | 1.8 |
| Flor Branca | 3.98 ^{c} | ±0.25 | 6.3 |
| Monami | 4.39 ^{b} | ±0.08 | 1.8 |
| Average | 4.30 | | |

*Nutrilite Acerola Super C
Different letters on columns indicate significant differences by the Tukey's test (p < 0.05).

### Example 3

### Fruit Firmness

Firmness was instrumentally measured at two opposite sides of 20 fruits, using a penetrometer, IP-90DI 200 Impac, using a 2.5 mm cylindrical probe and results were expressed in Newton (N).

### Juice Yield

In order to obtain the yield, the acerola cherry fruits were chopped in multiprocessor, model XL, (Philips Walita®). The pulp samples obtained from each genotype were centrifuged at 1050×g for seven minutes in order to separate the pulp by decanting the residue from the juice. The results for juice yield were expressed as a mass percentage of juice mass per fruit mass.

Table 3 shows the fruit firmness results ranged from 9.99 to 20.5 N. Flor Branca had the greatest fruit firmness among the evaluated genotypes, 20.5 N. Values below the total average were obtained in genotypes A, C, F, D, and BRS 366. Flor Branca showed the best result for juice yield, 71.3% followed by followed by the cultivar Monami, 68.3%.

TABLE 3
Analysis of Variance of Acerola Genotype Fruit Firmness and Juice
Yields (Ubajara, Ceará, 2017)

| | Fruit Firmness (N)^{†} | | Juice Yield (%) | |
|---|---|---|---|---|
| Genotype | Mean | SD | Mean | SD |
| A* | 99 ^{j} | ±1.8 | 63.5 ^{d} | ±1.73 |
| B | 15.1 ^{e} | ±1.42 | 64.3 ^{c} | ±0.96 |
| C | 11.7 ⁱ | ±1.13 | 61.3 ^{d} | ±1.50 |
| D | 12.9 ^{f} | ±1.11 | 63.5 ^{d} | ±0.60 |
| E | 180 ^{b} | ±0.96 | 67.8 ^{b} | ±3.10 |
| F | 11.8 ^{h} | ±2.01 | 64.8 ^{c} | ±0.50 |
| BRS 235 | 16.8 ^{c} | ±1.70 | 64.8 ^{c} | ±0.50 |
| BRS 366 | 12.7 ^{g} | ±2.77 | 63.8 ^{d} | ±0.50 |
| Flor Branca | 20.5 ^{a} | ±2.57 | 71.3 ^{a} | ±0.50 |
| Monami | 16.8 ^{d} | ±1.32 | 68.3 ^{b} | ±3.30 |
| Average | 14.6 | | 65.3 | |

*Nutrilite Acerola Super C
^{†}Firmness of green fruit
Different letters on columns indicate significant differences by the Tukey's test (p < 0.05)

The phenotypic results obtained revealed that there is potential among the new genotypes for a breeding program. The Nutrilite Acerola Super C and Monami exhibited high productivity (kg·tree⁻¹) during the studied period and overcame cultivar BRS 366. Cultivar BRS 366 is the reference and is widely used in commercial orchards due to high vitamin C yield per hectare (Freire, 2012). The productivity performance comparison suggest that Nutrilite Acerola Super C is at least equivalent to commercial genotypes. It was also observed that BRS 235 showed good productivity, with a similar result to that found in the Apodi region in a previous study (Paiva, 2003). The Nutrilite Acerola Super C showed highest production, 30.3 tons. ha⁻¹ with a density of 600 trees·ha⁻¹. This productivity exceeded the results obtained at Petrolina, Pernambuco, considered an international reference in the production of this fruit, producing annually 20 tons·ha⁻¹ (Martins et al., 2016).

Fruit firmness is one of the desired characteristics in the cultivar selection even when harvesting in the green stage. Cultivars with high fruit firmness are useful for orchards that use mechanized harvesting because the fruits typically suffer fewer injuries through the physical impacts. The Flor Branca cultivar had the highest firmness, 20.5 N, indicating that fruit from this cultivar may have a longer post-harvest life. Despite the best performance in productivity and vitamin C, Nutrilite Acerola Super C had the lowest fruit firmness (9.9 N). This is an important consideration for use in mechanized harvesting. When evaluating ripe fruits from 45 acerola clones, the overall firmness mean was 3.59 N, and the highest firmness was 6.48 N (Moura et al., 2007). The results found in the study were higher, an outcome already expected, since the evaluations were made in green fruits.

Considering the juice yield used in vitamin C industry, that is, the juice obtained after the removal of residue present in the pulp, the variation was 61.3% to 71.3%. The cultivar Monami and Flor Branca, maintained the best results, with 68.3% and 71.3% respectively. Lower results were found in green fruits collected at Embrapa Agroindústria Tropical, Pacajús, Ceará, with yields of 57% (Righetto, 2003), probably due to genotypic, environmental, and management influences, mainly related to the rainy season or irrigation. Another study shows a variation of 61.36% to 86.18% in mature fruits from fourteen genotypes evaluated in Londrina Paraná state (Carpentieri-Pípolo et al., 2000).

### Example 4

### Bioactive Compounds

The total vitamin C was determined by titration with 0.02% DFI, 2,6-dichloro-indophenol (Strohecker and Henning, 1967). Acerola pulp samples (0.5 g) were diluted to 100 mL in 0.5% oxalic acid and homogenized using an Ultra-Turrax blender (T25, Ika Works Inc., USA). Then, 5 mL of this solution was diluted to 50 mL with distilled water and titrated. The results were expressed as mg·100 g⁻¹ of pulp.

The same acerola extract was used to conduct the total phenol and total antioxidant activity, the samples extracted in 50% methanol and 70% acetone (Larrauri et al., 1997). The total phenol content was measured by a colorimetric assay using Folin-Ciocalteu reagent as described by (Ainsworth and Gillespie, 2007). Extracts were added to 1 mL Folin-Ciocalteau reagent (1 N), 2 mL of 20% Na₂CO₃, and 2 mL of distilled water. Absorbance at 700 nm was measured and the results were calculated based on a standard curve of 98% gallic acid (0-50 μg) and expressed as gallic acid equivalents (GAE) mg·100 g⁻¹ of pulp.

The total antioxidant activity (TAA) was determined using the 2,2-azinobis-3-ethylbenzthiazoline 6-sulphonic acid radical cation (ABTS*⁺) method (Rufino et al., 2006). The reaction was started by adding 30 μL of acerola extract in 3 mL of radical solution, absorbance was measured (734 nm) after 6 min, and the decrease in absorption was used to calculate the total antioxidant activity (TAA). A calibration curve was prepared and different trolox concentrations ranging from 100 to 2,000 μM were evaluated against the radical. Antioxidant activity was expressed as trolox equivalent antioxidant capacity (TEAC), i.e., μmol TEAC·g⁻¹ of pulp.

Acerola clones showed high divergence of vitamin C content, 2315.4 to 3251.6 mg·100 g⁻¹ of pulp. The highest significant content of vitamin C was observed in the Monami, 3252 mg·100 g⁻¹ and in the Nutrilite Acerola Super C, 3156 mg·100 g⁻¹, followed by BRS 235, with 3108 mg·100 g⁻¹, without differing between them. The lowest levels were found in genotypes D, E, BRS 366 and genotype F. The total phenols values obtained ranged from 2436.4 to 4208.7 mg GAE·100 g⁻¹ and the overall mean of 3402.53 mg GAE·100 g⁻¹). Nutrilite Acerola Super C showed the highest total soluble phenols (TSP) content, and was significantly higher than the other clones, followed by Monami, 4032.9 mg of GAE·100 g⁻¹ and BRS 235, 3998 mg of GAE·100 g⁻¹. Flor Branca cultivar obtained values above the average, 3884.4 mg of GAE·100 g⁻¹, but differed from Monami and BRS 235. The lowest values were observed in clones E, F and D, with 2743.5, 2667.6 and 2436.4 mg GAE·100 g⁻¹, respectively. The results of Total Antioxidant Activity (TAA) ranged from 327.54 to 119.32 μmol TEAC·g⁻¹ of pulp among acerola genotypes. The highest result was found in the Nutrilite Acerola Super C followed by Monami and BRS 235, with 306.80 and 297.91 μmol TEAC·g⁻¹ of pulp, respectively.

The content of total vitamin C, total soluble phenols (TSP) and total antioxidant activity (TAA) of acerola clones are shown in Table 4.

TABLE 4
Analysis of Variance of Acerola Genotype Bioactive Compounds and Total Antioxidant Activity
(Ubajara, Ceará, 2017)

| | Vitamin C | | TSP | | TAA (μmol | |
|---|---|---|---|---|---|---|
| | (mg · 100 g⁻¹) | | (mg GAE · 100 g⁻¹) | | TEAC · g⁻¹ of pulp) | |
| | | CV | | CV | | CV |
| Genotypes | Mean | (%) | Mean | (%) | Mean | (%) |
| A* | 3156 ^{a} | 0.7 | 4209 ^{a} | 1.4 | 327.5 ^{a} | 2.7 |
| B | 2820 ^{c} | 3.0 | 3497 ^{d} | 1.0 | 194.4 ^{e} | 2.2 |
| C | 2802 ^{e} | 0.5 | 3132 ^{e} | 1.3 | 198.3 ^{e} | 1.6 |
| D | 2376 ^{e} | 1.0 | 2436 ^{g} | 0.9 | 136.9 ^{f} | 6.6 |
| E | 2315 ^{e} | 3.0 | 2744 ^{f} | 0.8 | 119.3 ^{g} | 1.2 |
| F | 2636 ^{d} | 5.2 | 2668 ^{f} | 0.3 | 204.9 ^{e} | 0.5 |
| BRS 235 | 3108 ^{a} | 1.3 | 3998 ^{f} | 1.6 | 297.9 ^{b} | 1.2 |
| BRS 366 | 2613 ^{d} | 0.7 | 3425 ^{d} | 1.4 | 258.9 ^{c} | 1.6 |
| Flor | 2944 ^{b} | 1.9 | 3884 ^{c} | 0.2 | 230.3 ^{d} | 0.8 |
| Branca | | | | | | |
| Monami | 3252 ^{a} | 3.3 | 4033 ^{b} | 0.7 | 306.8 ^{b} | 0.6 |
| Average | 2802.2 | | 3402.6 | | 227.5 | |

*Nutrilite Acerola Super C
TSP: Total Soluble Phenols. TAA: Total Antioxidant Activity
Different letters on columns indicate significant differences by the Tukey's test (p < 0.05).

Monami, Nutrilite Acerola Super C, and BRS 235, showed the highest content of vitamin C followed by Flor Branca. The cultivar BRS 366, one of the main cultivars among the commercial orchards produced intermediary levels of vitamin C. This is similar to what was found in a previous study using immature fruits (Souza et al., 2014). However, BRS 366 was the most productive in terms of bioactive compounds and total antioxidant activity when compared with Florida Sweet and Flor Branca genotypes (Souza et al., 2014). The present study found that the most productive genotypes were Monami, Nutrilite Acerola Super C, and BRS 235 for vitamin C production. Nutrilite Acerola Super C had the highest Total Soluble Phenols and Total Antioxidant Activity and the second highest vitamin C production.

### Example 5

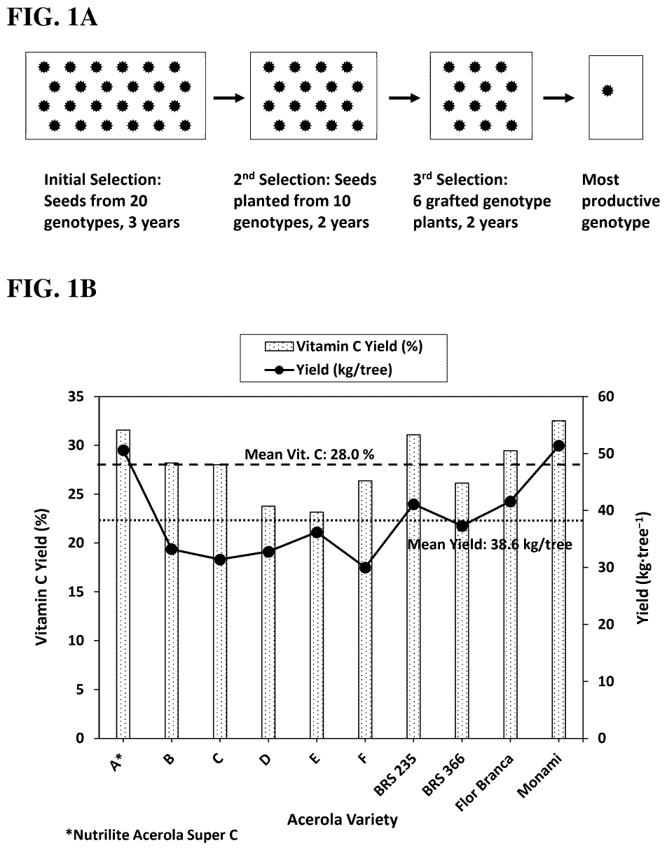

The vitamin C yield and fruit yield for the six genotypes and four cultivars were compared. Data for vitamin C yield from Table 4 are converted to percentages. Yield data are from the 2017 year in Table 1. These data are plotted in FIG. 1B.

TABLE 5
Comparison of Vitamin C and Fruit Yield from Genotypes and
Cultivars (Ubajara, Ceará, 2017)

| | Vitamin C | Yield |
|---|---|---|
| Genotype | (%) | (kg · tree⁻¹) |
| A* | 31.56 | 50.6 |
| B | 28.20 | 33.2 |
| C | 28.02 | 31.4 |
| D | 23.76 | 32.8 |
| E | 23.15 | 35.2 |
| F | 26.36 | 30.0 |
| BRS 235 | 31.08 | 41.1 |
| BRS 366 | 26.13 | 37.3 |
| Flor Branca | 29.44 | 41.6 |
| Monami | 32.52 | 51.4 |
| Average | 28.0 | 50.6 |

*Nutrilite Acerola Super C

### Example 6

### Genomic DNA Extraction

DNA was extracted using 50 mg of leaves from each genotype using Qiagen DNeasy Plant Kit (Qiagen, Hilden, Germany) as instructed by the manufacturer. DNA concentration was determined by spectrophotometry using the GTA 97 spectrophotometer (Global Equipment, São Paulo, Brazil), assuming an equivalence of 50 mg·mL⁻¹ per unit of absorbance at 260 nm. DNA samples were diluted to a final concentration of 30 ng·μL⁻¹.

Quality was determined by the absorbance ratio A₂₆₀ nm/A₂₈₀ nm and evaluated by electrophoresis on a 0.8% agarose gel in 1×TBE (90 mM Tris-base, 90 mM boric acid, and 2 mM EDTA) using DNA Lambda Hind III, (Invitrogen, Carlsbad, CA) molecular size marker was used as reference. The gels were stained with SYBR® Green (Sigma-Aldrich) and photographed under a UV transilluminator (KASVI, Curitiba, Brazil).

### Random Amplification of Polymorphic DNA (RAPD)

Forty primers from kits A and AX (Eurofins®) were tested in order to select the most informative ones (Table 6).

TABLE 6
Primer Sequences used in RAPD analysis

| Primer Name | Sequences (5′→3′) |
|---|---|
| OPA-04 | AATCGGGCTG (SEQ ID NO: 1) |
| OPA-08 | GTGACGTAGG (SEQ ID NO: 2) |
| OPA-20 | GTTGCGATCC (SEQ ID NO: 3) |
| OPAX-02 | GGGAGGCAAA (SEQ ID NO: 4) |
| OPAX-05 | AGTGCACACC (SEQ ID NO: 5) |
| OPAX-06 | AGGCATCGTG (SEQ ID NO: 6) |

The amplification mixture contained 1×PCR buffer (1.5 mM MgCl₂) (Eurofins®), 1.25 U Taq DNA polymerase (Go Taq®, Promega), 0.2 mM primer (Eurofins®), 2 mM of dNTPs (Eurofins®), 30 ng genomic DNA, and the final volume completed with nuclease-free water (Sigma-Aldrich) in a total reaction volume of 30 μL. The amplification was carried out in a C1000 Touch thermocycler (BIORAD, Hercules, CA, USA) with the following program: 38 cycles of 94° C. for 60 sec; 38° C. for 105 sec; 72° C. for 120 sec, followed by a final step of 72° C. for 7 min (Dias et al., 2015). The PCR products were electrophoresed on 1.5% agarose gels in 1×TBE (90 mM Tris-base, 90 mM boric acid, and 2 mM EDTA), stained with SYBR® Green (Sigma-Aldrich), and photographed under a UV transilluminator (KASVI, Curitiba, Brazil). Three replicates were tested for each sample. DNA molecular size marker 1,000 kb Ladder was obtained from Invitrogen (Carlsbad, CA).

The genetic similarity was determined using amplification products scored for the presence (1) and absence (0) of bands across the acerola genotypes in order to obtain a binary data matrix. Genetic similarities for RAPD data were calculated using Jaccard's similarity coefficient (Jaccard, 1908) and the Unweighted Pair Group Method with Arithmetic average (UPGMA) was used to build the dendrogram performed by software PAST v3.15 (Hammer, 2001).

### Genetic Similarity Matrix and Cluster Analysis

About 60 clear bands and 58 polymorphic bands were generated using the seven primers. The number of bands obtained for each primer ranged from three (OPA 08) to thirteen (OPAX02) with a mean of 8.57 bands per primer. The percentage of polymorphic bands was 97% and the highest number of polymorphic loci was 13 (100%) obtained with primer OPAX02 and lowest was seven (77.7%) with OPA 08, (Table 7).

TABLE 7
Primer sequence, total number of polymorphic bands, % of
polymorphism from seven primers used on characterization of 10
acerola genotypes, M. emarginata DC.

| | Sequences | | Polymorphic | Polymorphism |
|---|---|---|---|---|
| Primer Name | (5′→3′) | Total Loci | Loci | (%) |
| OPA 04 | AATCGGGCTG | 9 | 9 | |
| OPA 08 | GTGACGTAGG | 3 | 3 | |
| OPA 20 | GTTGCGATCC | 9 | 9 | |
| OPAX 02 | GGGAGGCAAA | 13 | 13 | |
| OPAX 05 | AGTGCACACC | 9 | 7 | |
| OPAX 06 | AGGCATCGTG | 8 | 8 | |
| OPAX 11 | TGATTGCGGG | 9 | 9 | |
| Total | | 60 | 58 | 97 |
| Mean | | 8.57 | 8.29 | |

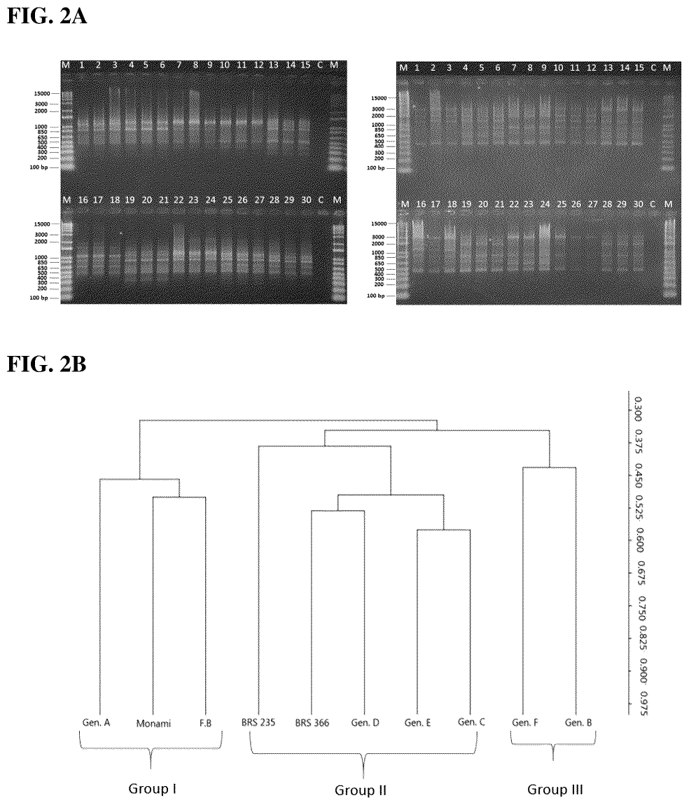

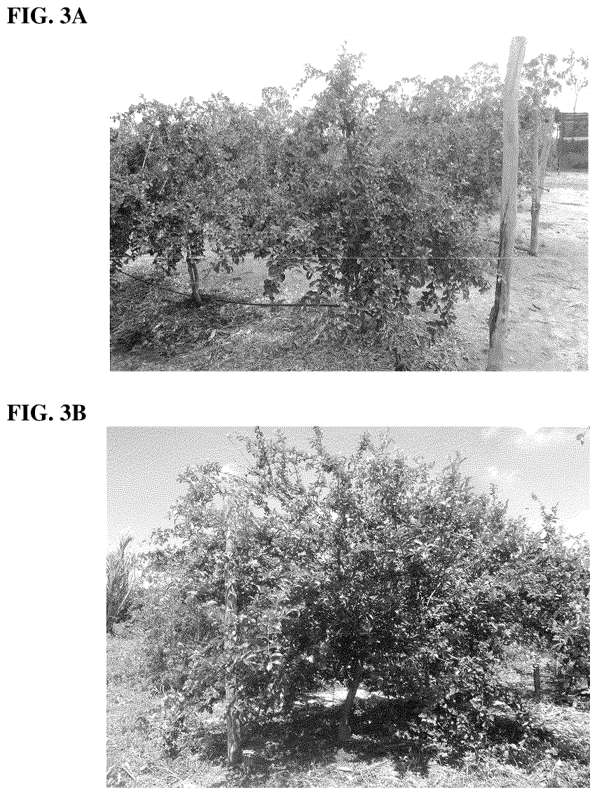

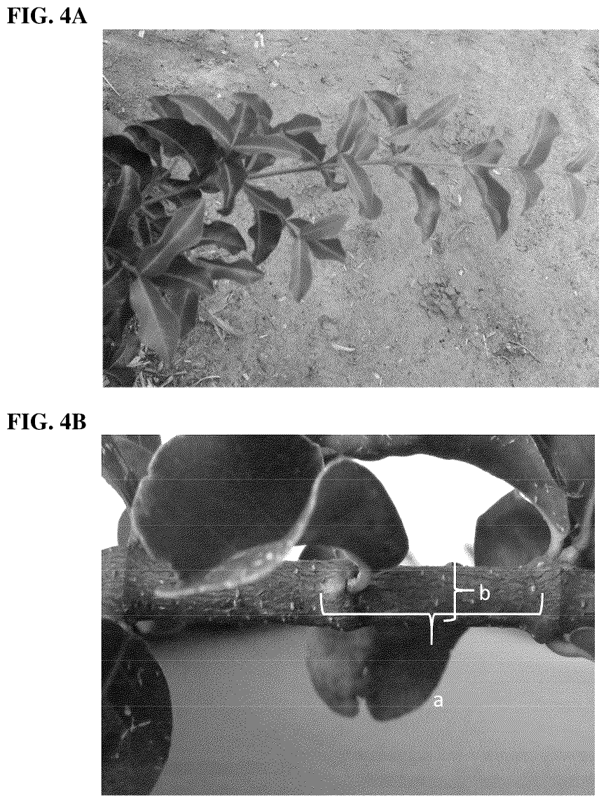

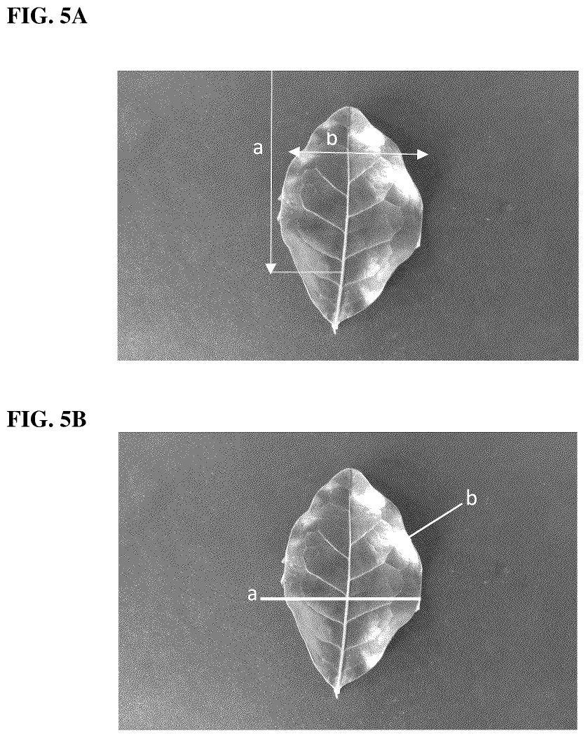

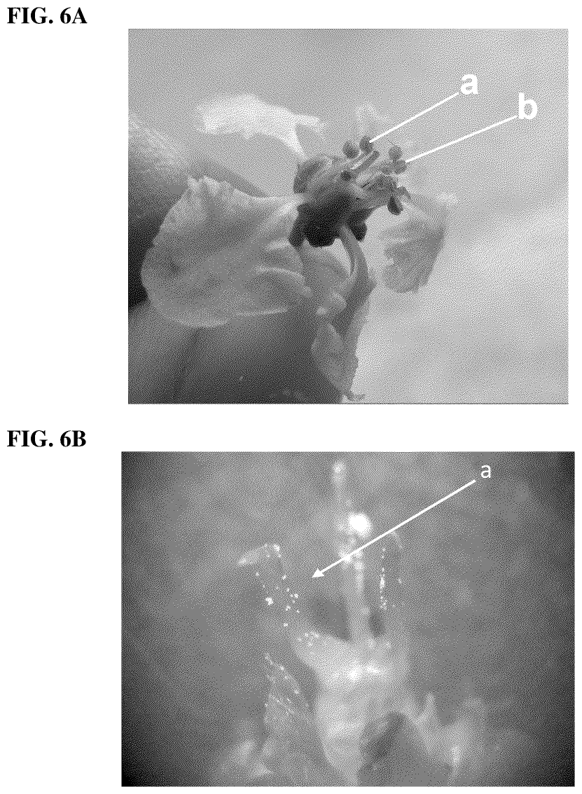

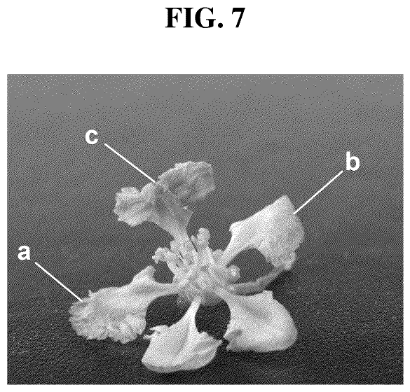

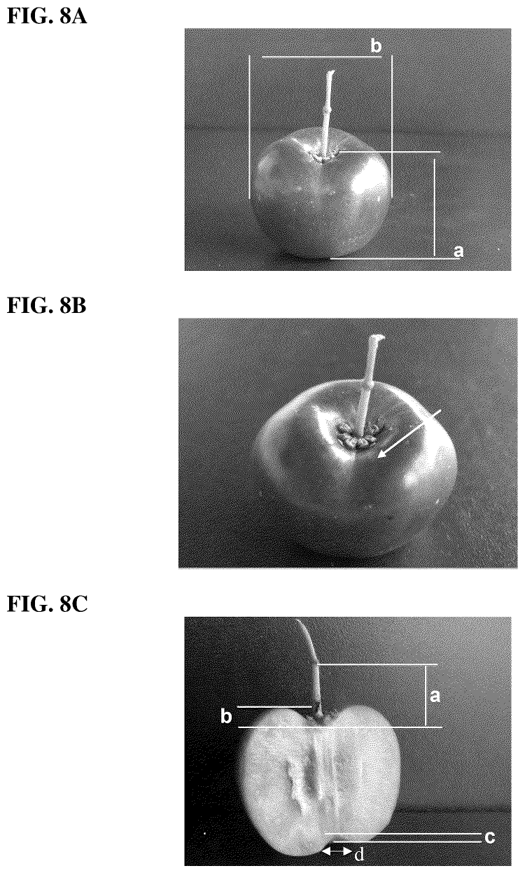

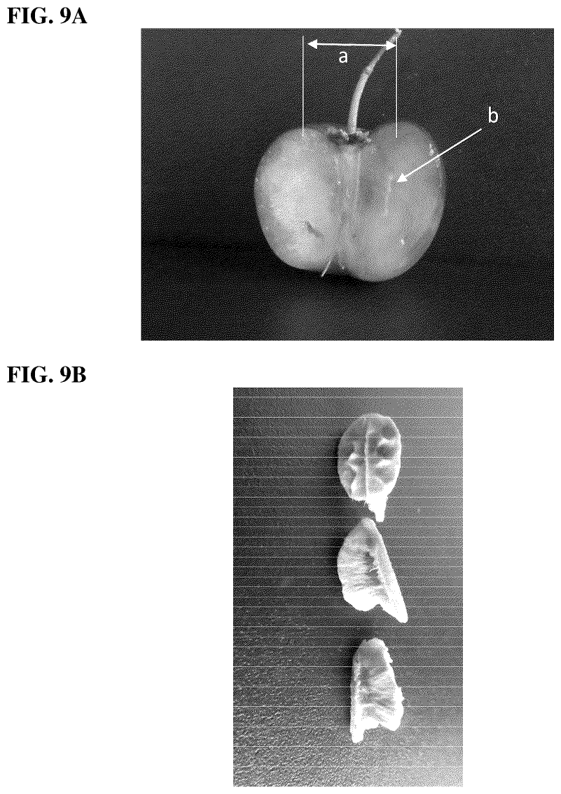

FIG. 2A shows the RAPD profile of six acerola genotypes and four cultivars using the random primer OPAX 02 (left side) and OPAX 05 (right side) (Eurofins®), by electrophoresis of 1.5% agarose gel, stained with SYBR® Green (Sigma-Aldrich), and visualized under UV transilluminator. M: molecular marker (DNA ladder 1 kb Plus, Invitrogen); 1-3 Monami; 4-6 BRS 366; 7-9 BRS 235; 10-12 Flor Branca; 13-15 Nutrilite Acerola Super C; 16-18 Genotype B; 19-21 Genotype C; 22-24 Genotype D; 25-27 Genotype E; 28-30 Genotype F; C Control.

Similarity coefficients for all the ten genotypes ranged from 0.20 (Genotypes E and Flor Branca) to 0.57 (Genotypes E and C), (Table 8).

TABLE 8
Jaccard's Similarity Coefficient Among Acerola Genotypes

| | BRS | BRS | | | | | | | | |
|---|---|---|---|---|---|---|---|---|---|---|
| Genotype | 235 | 366 | F. Branca | Monami | A | B | C | D | E | F |
| A* | 0.27 | 0.42 | 0.45 | 0.47 | 1.00 | 0.36 | 0.37 | 0.28 | 0.29 | 0.31 |
| B | 0.33 | 0.33 | 0.34 | 0.36 | 0.36 | 1.00 | 0.32 | 0.23 | 0.26 | 0.43 |
| C | 0.44 | 0.53 | 0.31 | 0.37 | 0.37 | 0.32 | 1.00 | 0.54 | 0.57 | 0.52 |
| D | 0.29 | 0.53 | 0.22 | 0.36 | 0.28 | 0.23 | 0.54 | 1.00 | 0.48 | 0.44 |
| E | 0.38 | 0.42 | 0.20 | 0.38 | 0.29 | 0.26 | 0.57 | 0.48 | 1.00 | 0.35 |
| F | 0.29 | 0.36 | 0.23 | 0.36 | 0.31 | 0.43 | 0.52 | 0.44 | 0.35 | 1.00 |
| BRS 235 | 1.00 | 0.42 | 0.29 | 0.28 | 0.27 | 0.33 | 0.44 | 0.29 | 0.38 | 0.29 |
| BRS 366 | 0.42 | 1.00 | 0.36 | 0.39 | 0.42 | 0.33 | 0.53 | 0.53 | 0.42 | 0.36 |
| F. Branca | 0.29 | 0.36 | 1.00 | 0.50 | 0.45 | 0.34 | 0.31 | 0.22 | 0.20 | 0.23 |
| Monami | 0.28 | 0.39 | 0.50 | 1.00 | 0.47 | 0.36 | 0.37 | 0.36 | 0.38 | 0.36 |

*Nutrilite Acerola Super C

The ten acerola genotypes were clustered into three main groups: Nutrilite Acerola Super C, Monami, and Flor Branca were in Group I; cultivar BRS 235, BRS 366, genotypes D and E were in Group II; and genotypes F and B were in Group III (FIG. 2A). FIG. 2B shows an unweighted pair group method with arithmetic mean (UPGMA) dendrogram of the acerola genotypes based on seven random RAPD primers and Jaccard's similarity coefficient.

The results obtained with RAPD markers showed a high level of polymorphism among the genotypes. The results corroborate with the variability found in the state of Pará, where 90.8% polymorphism was observed in 24 acerola genotypes (Salla et al., 2002). Polymorphism values closer to the present study were found in the active germplasm bank of the Federal University of Pernambuco with 94.51% among 42 accessions evaluated (Moraes Filho et al., 2013). The genetic diversity among acerola cultivars from the germplasm bank of Embrapa Semiárido, Petrolina, Pernambuco, presented 37.75% of polymorphism using ISSR (Inter Simple Sequence Repeats) markers (Souza et al., 2017). RAPD markers allowed greater access to genotype genetic variability than ISSR markers in cowpea (Vigna unguiculata) accessions (Dias et al., 2015).

### Example 7

### Association Among RAPD Markers and Phenotypic Traits

It is known that molecular markers are able to detect the genetic divergence among acerola genotypes and may contribute to the selection of clones in breeding programs. However, this method also requires phenotypic data to support the selection of superior candidates for clonal propagation (Lima et al., 2015). RAPD diversity among different eggplant varieties was useful in breeding programs for the development of genotypes resistant to Phomopsis disease, associating biotechnological tools with traditional breeding methods (Asad et al., 2015). Evaluating the genetic diversity in garlic germplasm was verified that morphological and molecular characterization formed similar groups, concluding based on results the importance of molecular characterization as a complementary technique, in order to strengthen the characterization by phenotypic descriptors (Viana et al., 2015). Likewise, comparing the dendrogram formed from RAPD markers (FIG. 2A) with phenotypic data, it is possible to observe associations among RAPD markers with agronomist characters desirable to select acerola cultivars.

Genotypes Nutrilite Acerola Super C, Monami, and Flor Branca had the best performance in productivity and vitamin C content each clustered together in Group I, suggesting that the obtained markers may be associated with genetic traits related to productivity and vitamin C content. BRS 235, the second best performing genotype (productivity and vitamin C content) clustered in proximity to Group I, but did not fall in the same group.

Group II, BRS 366, genotype D and E were clustered by productivity, since these genotypes did not show statistical difference among them, except BRS 235 and genotype C. Regarding the vitamin C results, there was a divergence between cultivar BRS 366 and genotype C. The association was observed only for the D and E genotypes that were statistically the same and remained in the same group.

Group III, genotype F and B, a similar result was observed with group II, where the genotypes were grouped by productivity, but no relation was observed with the results of vitamin C content.

Associations in terms of fruit firmness, total soluble phenols, and antioxidant activity was not reflected on the RAPD dendrogram. This may have been because the RAPD primers were arbitrary and non-specific to phenotypic traits.

Collectively, Nutrilite Acerola Super C and cultivar Monami presented the highest values of productivity, vitamin C, polyphenols, and antioxidant activity in comparison of the other groups. Results suggest that Nutrilite Acerola Super C is at least equivalent to commercial genotypes for productivity and vitamin C levels. The RAPD molecular markers showed high genetic diversity with 97% polymorphism among the acerola clones. The molecular markers identified allowed the distribution of the genotypes in three groups. When these results are compared with phenotypic data, a clear association among the productivity of the acerola trees was observed. There was also some association with vitamin C content for the most productive genotypes. These results show that RAPD markers are useful in evaluating breeding programs.

### Example 8

### Variety Description

The Nutrilite Acerola Super C acerola cherry (Malpighia emarginata DC.) variety (Genotype A), a representative sample of seed having been deposited under ATCC Patent Deposit Designation, Patent Deposit No. PTA-126522, was observed to possess the following morphological and physical characteristics, based on the average of 20 individual observations conducted at Amway Nutrilite Brazil farm in Ubajara, Ceará, Brazil during the 2017 growing season (Table 9).

TABLE 9
Nutrilite Acerola Super C Variety Description

| | |
|---|---|
| Characteristic* | Nutrilite Acerola Super C |

Tree

| | |
|---|---|
| Growth habit | Open (2) |

Branch (one year old)

| | |
|---|---|
| Branch density | High (7) |
| Internode length | Short (3) |
| Diameter | Medium (2) |
| Pubescence | Medium (2) |

Leaves

| | |
|---|---|
| Length | Medium (5) |
| Position of the widest part | Middle (2) |
| Margin | Medium (3) |
| Variegation | Absent (1) |
| Color Intensity | Dark (4) |

Flower

| | |
|---|---|
| Stigma position relative to anthers | Same level (2) |
| Curve of flower stiletto | Strongly Curved (3) |

Flower Petals

| | |
|---|---|
| Margin | Medium (3) |
| Color Intensity | Dark Pink (3) |

Fruit

| | |
|---|---|
| Length | Long (6) |
| Diameter | Big (7) |
| Ratio of Length to Diameter | Medium (2) |
| Mass (weight) | High (7) |
| Shape | Flattened (3) |
| Furrow depth | Deep (3) |
| Depth of distal cavity | Medium (2) |
| Distal cavity width | Narrow (1) |
| Depth of peduncular cavity | Medium (2) |
| Length of stem | Medium (5) |
| Peduncular cavity width | Deep (3) |
| Main Color | MR |
| Pulp color | Orange (2) |
| Acidity | High (4) |
| Succulence | Medium (5) |

Seed

| | |
|---|---|
| Size | Medium (2) |
| Color intensity | Medium (2) |

Agronomic Properties†

| | |
|---|---|
| Productivity (Year 1) (kg · tree⁻¹) | 28.86 (14.1%) |
| Productivity (Year 2) (kg · tree⁻¹) | 50.6 (4.01%) |
| Productivity (Year 1) (tons · hectare⁻¹) | 17.3 |
| Productivity (Year 2) (tons · hectare⁻¹) | 30.3 |
| Fruit mass (g) | 4.38 |
| Fruit Firmness (N) | 9.9 ± 1.8 |
| Juice Yield (% mass) | 63.5 ± 1.73 |
| Vitamin C (mg · 100 g⁻¹) | 3156 (0.7%) |
| Vitamin C (%) | 31.6% |
| Total soluble phenols | 4209 (1.4%) |
| (mg GAE · 100 g⁻¹) | |
| Total antioxidant activity | 327.5 (2.7%) |
| (μmol TEAC · g⁻¹ pulp | |

*Results are mean values obtained from the observation of 20 individual plants.
†Results are mean values obtained from four replicate experiments.

A comparison of the morphological characteristics of the acerola genotypes and cultivars is provided in Table 10.

TABLE 10
Morphological Comparison of Acerola Genotypes (Ubajara, Ceará, 2017)

| | Genotypes | | | | | | | | | |
|---|---|---|---|---|---|---|---|---|---|---|
| Morph. | | | | | | | BRS | BRS | F. | |
| characteristic | Gen. A* | Gen. B | Gen. C | Gen. D | Gen. E | Gen. F | 235 | 366 | Branca | Monami |
| Growth Habit | Open | Open | Open | Open | Straight | Open | Open | Open | Open | Straight |
| Branch Density | High | Medium | Low | Medium | Medium | High | Low | High | High | Medium |
| Internode | Short | Long | Long | Short | Short | Short | Long | Medium | Short | Medium |
| Length | | | | | | | | | | |
| Leaf Blade | Medium | Medium | Medium | Medium | Medium | Long | Medium | Medium | Short | Short |
| Length | | | | | | | | | | |
| Margin | Medium | Weak | Medium | Medium | Weak | Weak | Strong | Strong | Medium | Weak |
| Variegation | Absent | Absent | Absent | Absent | Absent | Absent | Absent | Absent | Absent | Absent |
| Green Color | Dark | Dark | Medium | Low | Dark | Medium | Dark | Low | Medium | Dark |
| Intensity | | | | | | | | | | |
| Flower Color | Dark | Dark | Medium | Medium | Medium | Medium | Medium | Medium | Light | Medium |
| Intensity | | | | | | | | | | |
| Fruit Shape | Flattened | Oval | Oblong | Flattened | Flattened | Oblong | Oblong | Circular | Circular | Circular |
| Fruit Main | MR | MR | MR | SR | SR | DR | DR | SR | SR | DR |
| Color | | | | | | | | | | |
| Pulp Color | Orange | Orange | Orange | Orange | Red | Red | Red | Orange | Red | Red |

Gen. A*: Nutrilite Acerola Super C; MR: Medium Red; SR: Soft Red; DR: Dark Red
*Results are mean values obtained from the observation of 20 individual plants.

### Example 9

### Nutrilite Acerola Super C Seed Production

Seeds of Nutrilite Acerola Super C were obtained for deposit by isolating a matrice (mother tree) within a shade house to ensure self-pollination and prevent cross-pollination with other varieties. Mature fruits were harvested about 25 days after flowering and pollination. The pulp was removed and the seeds were isolated and dried under ambient conditions. The seeds were then packaged in sealable plastic bags and stored at ambient conditions for up to 2 years.

### Nutrilite Acerola Super C Genotype Evaluation

Seeds obtained from Nutrilite Acerola Super C will be germinated to obtain seedlings with leaves. DNA will be extracted from the leaves and evaluated for polymorphism as described above. The results are expected to show genetic identity among multiple seed samples from Nutrilite Acerola Super C.

### Biological Deposits

A representative sample of seeds of acerola cherry (Malpighia emarginata) variety Nutrilite Acerola Super C was deposited with the American Type Culture Collection (ATCC), 10801 University Blvd., Manassas, Virginia, 20110, United States of America on Feb. 28, 2020 and was assigned ATCC Patent Deposit Designation, Patent Deposit No. PTA-126522, on Mar. 2, 2020. The deposit will be maintained at the ATCC depository under the terms of the Budapest Treaty on the International Recognition of the Deposit of Microorganisms for the Purposes of Patent Procedure for a term of at least thirty years and at least five years after the most recent request for the furnishing of a sample of the deposit was received by the depository. Applicants have satisfied all the requirements of 37 C.F.R. §§ 1.801-1.809, including providing an indication of the viability of the sample. Additional deposits will be made at the ATCC as needed to ensure availability, subject to the conditions described herein. Applicants impose no restrictions on the availability of the deposited material from the ATCC after the issuance of a patent from this application. Applicants have no authority to wave any restrictions imposed by law on the transfer of biological material or its transportation in worldwide commerce. Applicants do not waive any of their rights granted under any patents issuing from this application in any country or under the U.S. Plant Variety Protection Act (7 U.S.C. § 2321 et seq.) or other international or foreign plant variety protection systems.

1. An acerola cherry (Malpighia emarginata DC.) variety designated Nutrilite Acerola Super C, a representative sample of seed having been deposited under ATCC Patent Deposit Designation, Patent Deposit No. PTA-126522.

2. The cherry of claim 1, wherein a cherry fruit comprises one or more of the characteristics:
fruit firmness of about 8 to 12 N;
juice yield of about 50-70%;
vitamin C content of about 3000-3500 mg·100 g⁻¹;
total soluble phenols of about 4000-4500 mg GAE·100 g⁻¹; or
total antioxidant activity of about 300-350 μmol TEAC·g⁻¹ pulp.

3. The cherry of claim 1, wherein a cherry fruit has an average mass of about 4.4 g.

4. A plant product produced from one or more of the acerola cherry variety of claim 1, comprising a cell or the genetic material of the cherry.

5. The plant product of claim 4, wherein the plant product comprises fruit, juice, pulp, extracts, vitamin C, polyphenols, antioxidants, derivatives thereof, or combinations thereof.

6. Acerola cherry juice or pulp produced from one or more of the acerola cherry variety of claim 1, comprising a cell or the genetic material of the cherry.

7. A food product, health supplement, or nutraceutical produced from one or more of the acerola cherry variety of claim 1, comprising a cell or the genetic material of the cherry.

8. A food product comprising acerola cherries or a plant product thereof of claim 1, comprising a cell or the genetic material of the cherry.

9. A seed of the acerola cherries of claim 1, comprising a cell or the genetic material of the cherry.

10. An acerola plant, or part thereof, produced by growing the cherry of claim 1, comprising a cell or the genetic material of the cherry.

11. The acerola plant of claim 10, wherein the acerola plant is produced by natural plant breeding and vegetative propagation.

12. The acerola plant of claim 10, wherein the plant yields about 25 to about 60 kg·tree⁻¹ or about 15 to about 36 metric tons of fruit per hectare.

13. A homogenous population comprising a plurality of the acerola plant of claim 10.

14. The acerola plant of claim 10, wherein the acerola plant comprises or confers to its seed one or more traits comprising greater fruit yield, greater fruit mass, greater productivity, greater amounts of vitamin C, greater amounts of polyphenols, greater amounts of antioxidants, improved fruit firmness, greater juice yields, increased protein yield, increased fiber yield, increased fertilizer utilization, improved adaptation to winter planting, increased drought tolerance, increased cold tolerance, increased photoperiod, herbicide tolerance, increased insect resistance, increased disease resistance, or combinations thereof, as compared to other varieties of acerola plants or wild-type acerola.

15. A tissue culture of cells produced from the acerola plant of claim 10, wherein the cells are produced from a plant part comprising embryo, meristematic cell, leaf, cotyledon, hypocotyl, root, root tip, stem, pistil, anther, ovule, flower, pollen, or seed.

16. An acerola cherry (Malpighia emarginata DC.) plant regenerated from the tissue culture of claim 15, wherein the acerola plant comprises the morphological and physiological characteristics of variety Nutrilite Acerola Super C, a representative sample of seed having been deposited under ATCC Patent Deposit Designation, Patent Deposit No. PTA-126522.

17. An acerola cherry (Malpighia emarginata DC.) plant or plant part of the variety designated Nutrilite Acerola Super C, a representative sample of seed having been deposited under ATCC Patent Deposit Designation, Patent Deposit No. PTA-126522.

18. A homogenous population comprising a plurality of the acerola plant of claim 17, wherein the population yields about 25 to about 60 kg·tree⁻¹ or about 15 to about 36 metric tons of fruit per hectare.

19. Fruit produced from the acerola plant of claim 17, comprising a cell or the genetic material of the cherry plant.

20. The fruit of claim 19, wherein the fruit comprises one or more of the characteristics:
fruit firmness of about 8 to 12 N;
juice yield of about 50-70%;
vitamin C content of about 3000-3500 mg·100 g⁻¹;
total soluble phenols of about 4000-4500 mg GAE·100 g⁻¹; or total antioxidant activity of about 300-350 μmol TEAC·g⁻¹ pulp.

21.-30. (canceled)


SEQUENCE LISTING (7 sequences)

SEQ ID NO: 1 - DNA, 10 bp, synthetic construct
  misc_feature 1..10: note = OPA-04 primer
  source 1..10: mol_type = other DNA
aatcgggctg

SEQ ID NO: 2 - DNA, 10 bp, synthetic construct
  misc_feature 1..10: note = OPA-08 primer
  source 1..10: mol_type = other DNA
gtgacgtagg

SEQ ID NO: 3 - DNA, 10 bp, synthetic construct
  misc_feature 1..10: note = OPA-20 primer
  source 1..10: mol_type = other DNA
gttgcgatcc

SEQ ID NO: 4 - DNA, 10 bp, synthetic construct
  misc_feature 1..10: note = OPAX-02 primer
  source 1..10: mol_type = other DNA
gggaggcaaa

SEQ ID NO: 5 - DNA, 10 bp, synthetic construct
  misc_feature 1..10: note = OPAX-05 primer
  source 1..10: mol_type = other DNA
agtgcacacc

SEQ ID NO: 6 - DNA, 10 bp, synthetic construct
  misc_feature 1..10: note = OPAX-06 primer
  source 1..10: mol_type = other DNA
aggcatcgtg

SEQ ID NO: 7 - DNA, 10 bp, synthetic construct
  misc_feature 1..10: note = OPAX-11 primer
  source 1..10: mol_type = other DNA
tgattgcggg